(19) United States
(12) Patent Application Publication
(10) Pub. No.: US 2002/0182222 A1
(43) Pub. Date: Dec. 5, 2002

(54) HIV vaccine candidate peptides

(76) Inventor: Anne De Groot, Providence, RI (US)

Correspondence Address: MINTZ, LEVIN, COHN, FERRIS,, GLOVSKY and POPEO, P.C., One Financial Center, Boston, MA, 02111, US

(21) Appl. No.: 10/055,524

(22) Filed: Oct. 26, 2001

Related U.S. Application Data
(62) Division of application No. 09/351,036, filed on Jul. 9, 1999.
(60) Provisional application No. 60/092,346, filed on Jul. 10, 1998.
(60) Provisional application No. 60/115,145, filed on Jan. 8, 1999.
(60) Provisional application No. 60/130,677, filed on Apr. 23, 1999.

(51) Int. Cl. A61K 48/00; A61K 39/21; A61K 39/29; C12N 9/22

(52) U.S. Cl. 424/188.1; 435/199; 514/44

(57) ABSTRACT

The invention provides HIV vaccine candidates that have “evolved” due to gene shuffling in vitro for inclusion of “cross-clade” characteristics. The invention also provides a method for identifying HIV vaccine candidates that could be presented in the context of more than one HLA, due to the creation of promiscuous epitopes by gene shuffling.

### CLAIM OF PRIORITY

This application claims priority under 35 U.S.C. §119(e) to U.S. provisional patent applications No. 60/092,346, filed Jan. 8, 1999; and No. 60/130,677, filed Apr. 23, 1999.

### STATEMENT REGARDING FEDERALLY SPONSORED RESEARCH

This invention was made with United States Government support from the National Institutes of Health. The Government may have certain rights in the invention.

### TECHNICAL FIELD OF THE INVENTION

This invention relates generally to vaccines, particularly to vaccines to human immunodeficiency virus 1 (HIV-1).

### BACKGROUND OF THE INVENTION

The need for an effective vaccine against human immunodeficiency virus type 1 (HIV-1), one that takes into consideration the variability of HIV strains, remains urgent. Researchers have yet to achieve the development of an HIV vaccine that will stimulate effective immune responses to most of the many different strains (“clades”) of HIV now being transmitted in course of the global HIV epidemic. At the root of the problem is the great diversity of HIV itself, and the restriction of human cytotoxic T cell (CTL) response to variant strains of HIV.

In the course of developing HIV vaccines, most researchers have focused on defining immune responses against a particular vaccine candidate. Most of these candidate vaccines in Phase I through Phase III trials at present belong to the group of lade B strains of HIV. Some of these vaccine candidates are derived from lab strains of HIV, others are derived from lade B patient isolates. “Challenge” strains of HIV, to which immunized individuals may be exposed, may be 10 to 15% different at the level of their sequences. Challenge strains in other regions of the world, and new strains arriving in the U.S. from other regions of the world may be even more dramatically divergent. These variations may allow the challenge strains to elude the vaccine-mediated CTL responses. In other words, due to strain variations, immune responses raised against one vaccine strain may not protect against other strains of HIV.

The root of this problem is the interaction between viral protein sequences and the molecules of the immune system (the human leukocyte antigens; HLA), whose duty it is to present peptides derived from the proteins of the challenge virus to the immune system and to engage vaccine-trained T cells to respond. Due to the tight-fit nature of the interaction between virus-derived peptides and the HLA, changes in amino acid sequence of a challenge strain may interfere with the ability of a given peptide to bind to the HLA molecule, preventing recognition of the challenge strain by T cell clones raised against a clade B vaccine construct. Sequence modifications at the amino acid level may affect the recognition of the epitope in three ways: (1) by affecting intracellular processing, (2) by interfering with binding (of the peptide) to major histocompatibility (such as major histocompatibility complex (MHC) or HLA) molecules and presentation of the peptide-HLA complex at the antigen presenting-cell surface, and (3) by interfering with binding of the epitope to the T cell receptor (TCR) (Germain & Margulies, 11 Ann. Rev. Immunol. 403 (1993); Falk et al., 351 Nature 290 (1991)). Thus, the impact of HIV variation at the molecular level may be to diminish cross-clade protection by a vaccine that does not contain CTL epitopes that are conserved across strains of HIV, or epitopes that are more representative of non-B clades.

Many studies of cross-clade recognition of HIV epitopes have been carried out (see, Wilson et al., 14(11) AIDS Res. Hum. Retroviruses 925-37 (1998); McAdam et al., 12(6) AIDS .571-9 (1998); Lynch et al., 178(4) J Infect Dis. 1040-6 (1998); 95 Dev. Biol. Stand. 147-53 (1998); Cao et al., 71(11) J. Virol. 8615-23 (1997); Durali et al., 72(5) Virol. 3547 53 (1998)). In general, these studies often used whole-gene, vaccinia-expressed constructs to probe CTL lines from HIV-1 infected or HIV-1 vaccinated volunteers for CTL responses. What appeared to be cross-clade recognition by CTL in these experiments, may have been recognition of CTL epitopes that are conserved within the large gene constructs cloned into the vaccinia constructs and into the vaccine strain (or the autologous strain). Where responses to specific peptides, and their altered sequences in other HIV strains, have been tested, and the peptides have been mapped, some studies have shown a lack of cross-strain recognition (Dorrel et al., HIV Vaccine Development Opportunities And Challenges Meeting, Abstract 109 (Keystone, Colo., January 1999)). Studies of virus escape from CTL recognition carried out on HIV-1 infected individuals have also shown that viral variation at the amino acid level may abrogate effective CTL responses (Koup, 180 J. Exp. Med. 779 (1994); Dai et al, 66 J. Virol. 3151 (1992); Johnson et al., 175 J. Exp. Med. 961 (1992)).

As yet, no single HIV strain has been found that will stimulate effective HLA-restricted immune response against a wide range of HIV strains. Thus, a need remains in the art for a “world lade” vaccine.

### SUMMARY OF THE INVENTION

The invention provides HIV vaccine candidate peptides, including the HIV peptides shown in any of FIG. 2 (SEQ ID NO: 1-27), TABLES 6-31 (SEQ ID NO:28-626); and FIGS. 6-9 and TABLE 14 (SEQ ID NO:627-672). The invention also provides an HIV vaccine, which is an HIV peptide in an immunologically acceptable excipient, such as any of the vaccine carriers known in the medical arts. In one aspect of the invention, the HIV vaccine candidates have “evolved” due to gene shuffling in vitro for inclusion of “cross-clade” characteristics.

The invention also provides a method for identifying HIV vaccine candidates that could be presented in the context of more than one HLA, due to the creation of promiscuous epitopes by gene shuffling. Cross-clade HIV peptides are identified. A “cross-clade” HIV peptides is an IRV peptide conserved across several HIV strains having different MHC binding potential. The HIV strains are likely to be presented by MHC molecules representing the most prevalent human HLA alleles. Next, the identified HIV peptides are analyzed for being putative ligands for HLA alleles. Then, HIV peptides that are putative ligands for highly prevalent HLA are as being HIV vaccine candidates. In one embodiment, the cross-clade HIV peptides belong to a consensus sequence obtained from the Los Alamos HIV Sequence Database.

### BRIEF DESCRIPTION OF THE DRAWINGS

FIG. 1 is a histogram showing the distribution of the number of HIV-1 isolates in which 8-mer to 11-mer peptides predicted to bind (A) and (b) HLA-B27 are exactly conserved.

FIG. 2 is a table showing the results for the 8-mer to 11-mer peptides for analysis. The second and third columns shows the estimated binding probability for peptides with EpiMatrix scores at least as high as these peptides. The fourth and fifth columns give the highest fold-change in MFI at any concentrations if over 1.3. The sixth column indicates whether the peptide has been published as a known epitope restricted to the appropriate allele. Parentheses indicate that the peptide is contained within an epitope of unknown restriction. The seventh column indicates the protein of origin. The eighth column indicates the number of isolate sequences containing this exact amino acid sequence. The ninth column indicates the approximate position of this ligand relative to the LAI reference strain. The tenth through fifteenth columns indicate whether any of the sequences in which the peptide is conserved are designated as belonging to clades A-E or other lade.

FIG. 3 is a description of the project outline for identifying regional HIV vaccine candidate peptides.

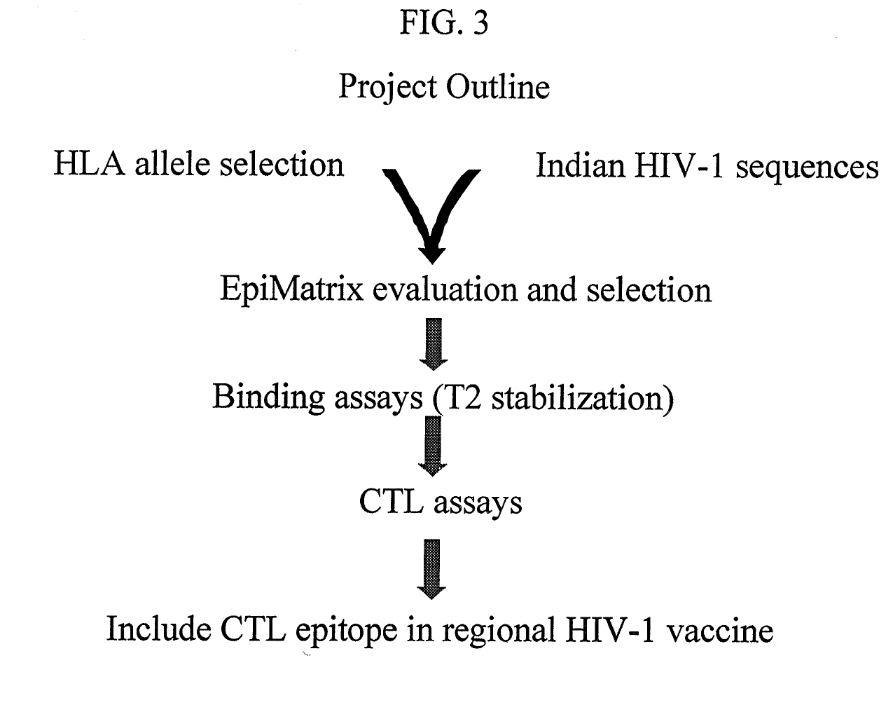

FIG. 4 is a pie chart showing the results of methods for HLA-A allele selection.

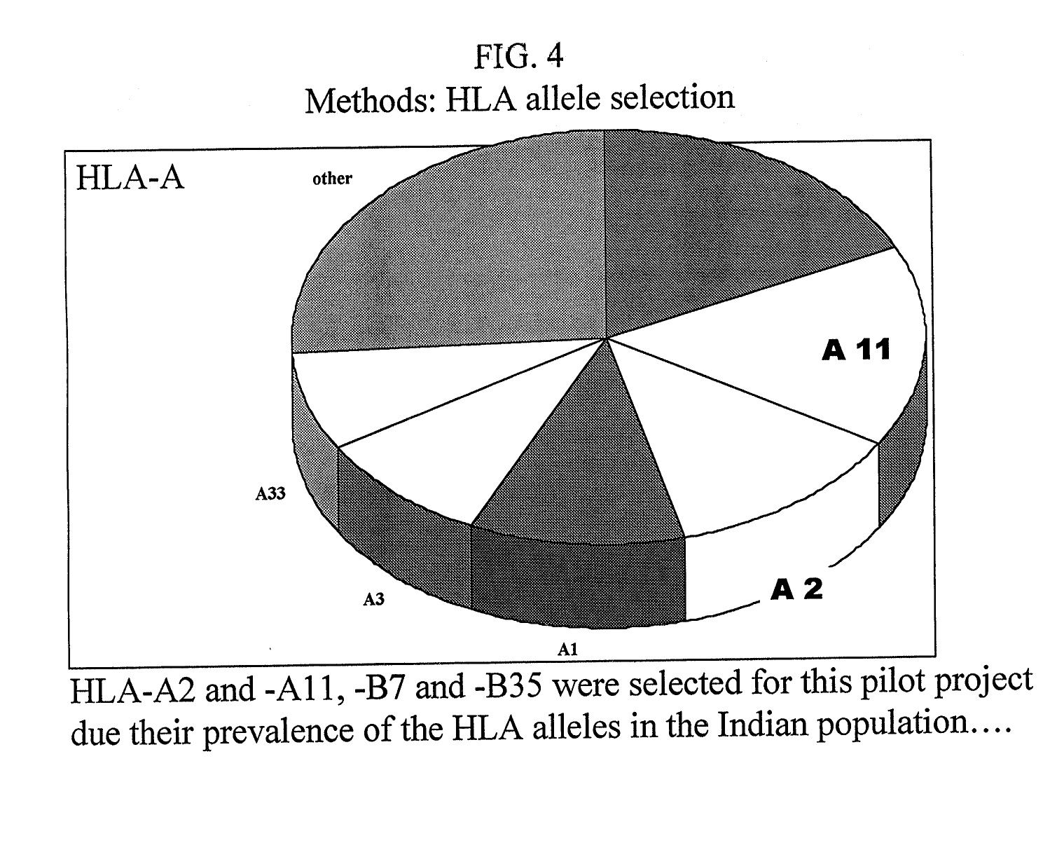

FIG. 5 is a pie chart showing the results of methods for HLA-B allele selection.

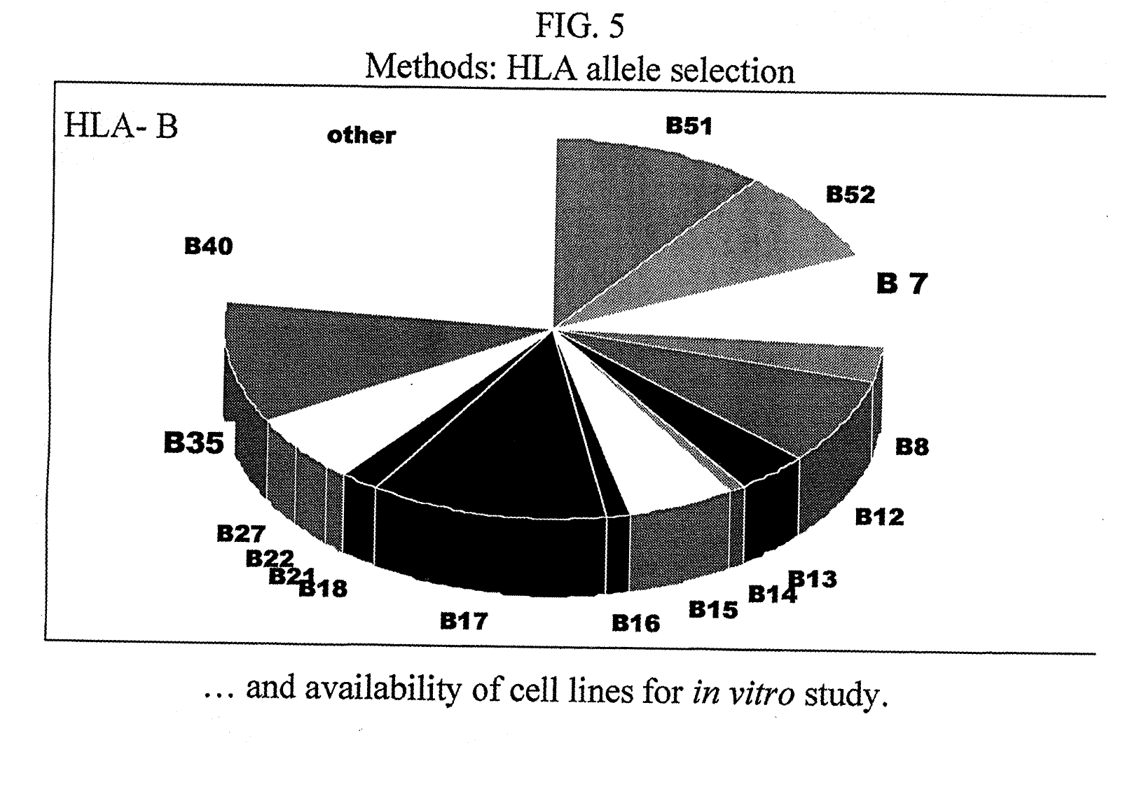

FIG. 6 is a table showing EpiMatrix predictions and binding results for B7.

FIG. 7 is a table showing EpiMatrix predictions and binding results for B37.

FIG. 8 is a table showing EpiMatrix predictions and binding results for A2.

FIG. 9 is a table showing EpiMatrix predictions and binding results for A11.

FIG. 10 is a description of the methods T2 binding assay.

FIG. 11 is a bar graph showing the clustering of putative MHC ligands in env. At left, the number of putative ligands discovered to be both conserved across clades and likely to bind to at least one human class I MHC is shown by location in a “consensus” sequence obtained from the Los Alamos HIV Sequence Database. This analysis demonstrates regions of distinct clustering. Such regions will be analyzed for representation of HLA alleles. Regions that contain clusters of putative ligands representing highly prevalent HLA were of interest for vaccine development.

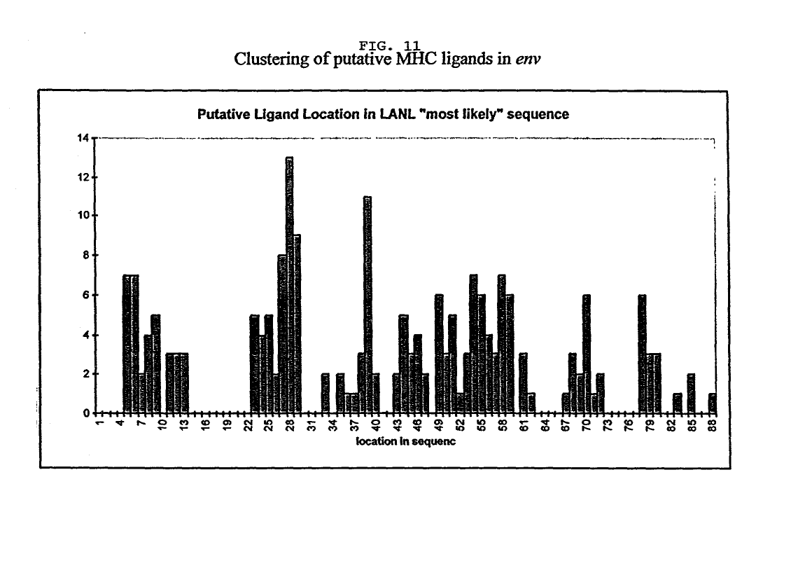

### DETAILED DESCRIPTION OF THE INVENTION

Vaccines can include any one of the HIV vaccine candidate peptides disclosed below, either alone, in combination with suitable carriers, linked to carrier proteins, or expressed from a polynucleotide, such as a “naked DNA” vaccine. The peptides can be administered to a host for treatment of HIV. The peptides can also be used to enhance immunologic function.

Peptides. The HIV vaccine candidate peptides can be produced by well known chemical procedures, such as solution or solid-phase peptide synthesis, or semi-synthesis in solution beginning with protein fragments coupled through conventional solution methods, as described by Dugas & Penney, Bioorganic Chemistry, 54-92 (Springer-Verlag, New York, 1981). For example, peptides can be synthesized by solid-phase methodology utilizing an PE-Applied Biosystems 430A peptide synthesizer (commercially available from Applied Biosystems, Foster City, Calif.) and synthesis cycles supplied by Applied Biosystems. Boc amino acids and other reagents are commercially available from PE-Applied Biosystems and other chemical supply houses. Sequential Boc chemistry using double couple protocols are applied to the starting p-methyl benzhydryl amine resins for the production of C-terminal carboxamides. After synthesis and cleavage, purification is accomplished by reverse-phase C18 chromatography (Vydac) column in 0.1% TFA with a gradient of increasing acetonitrile concentration. The solid phase synthesis could also be accomplished using the FMOC strategy and a TFA/scavenger cleavage mixture.

When produced by conventional recombinant means, (described below) the HIV vaccine candidate peptide can be isolated either from the cellular contents by conventional lysis techniques or from cell medium by conventional methods, such as chromatography (see, e.g., Sambrook et al., Molecular Cloning. A Laboratory Manual., 2d Edition (Cold Spring Harbor Laboratory, New York (1989).

The general construction and use of synthetic HIV peptides is disclosed in U.S. Pat. Nos. 5,817,318 and 5,876,731, the contents of which are incorporated by reference.

In one embodiment, the HIV vaccine candidate peptide has a maximum size of 50 amino acids in length and a minimum size of 8 amino acids (for the relevant SEQ ID NOS) to 11 amino acids (for other relevant SEQ ID NOS). The peptide can be any size between the minimum to maximum size, and one HIV vaccine candidate peptide can be of a given size independently of another HIV vaccine candidate peptide. For example one HIV vaccine candidate peptide can be 25 amino acids in length while another HIV vaccine candidate peptide is 45 amino acids in length.

Peptides as antigens. The HIV vaccine candidate peptides are useful as antigens for raising anti-HIV immune responses, such as T cell responses (cytotoxic T cells or T helper cells). An “antigen” is a molecule or a portion of a molecule capable of stimulating an immune response, which is additionally capable of inducing an animal or human to produce antibody capable of binding to an epitope of that antigen. An “epitope” is that portion of any molecule capable of being recognized by and bound by an MHC molecule and recognized by a T cell or bound by an antibody. An antigen can have one or more than one epitope. The specific reaction indicates that the antigen will react, in a highly selective manner, with its corresponding MHC and T cell, or antibody and not with the multitude of other antibodies which can be evoked by other antigens.

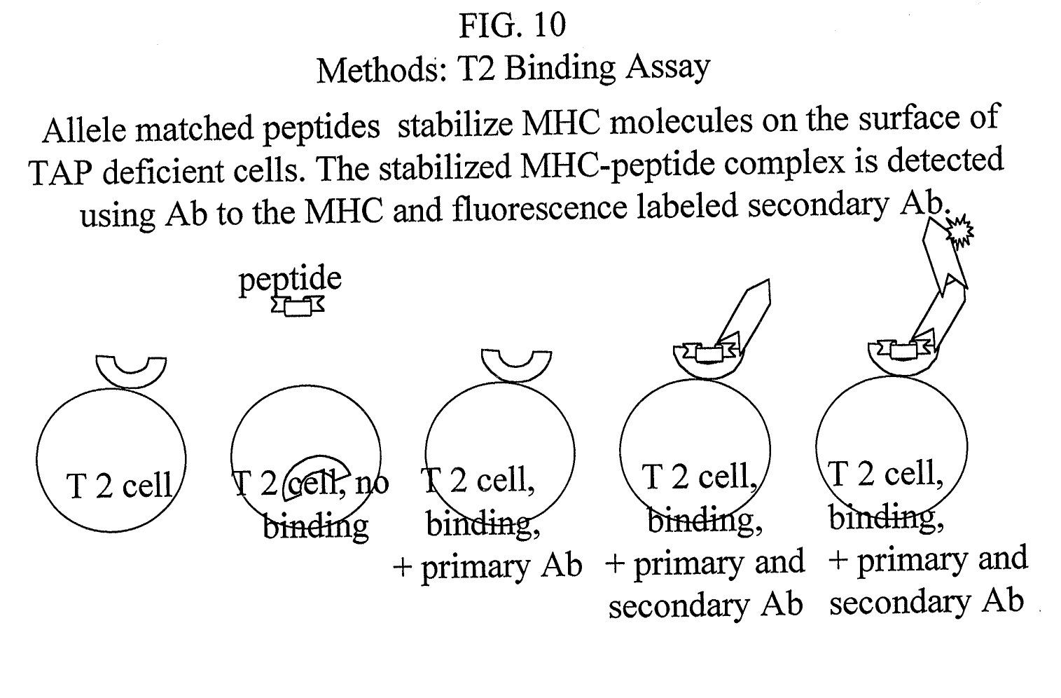

A peptide is “immunologically reactive” with an T cell or antibody when it binds to an MHC and is recognized by a T cell or binds to an antibody due to recognition (or the precise fit) of a specific epitope contained within the peptide. Immunological reactivity can be determined by measuring T cell response in vitro or by antibody binding, more particularly by the kinetics of antibody binding, or by competition in binding using as competitors a known peptides containing an epitope against which the antibody or T cell response is directed. The techniques for determining whether a peptide is immunologically reactive with an T CELL or with an antibody are known in the art. The peptides can be screened for efficacy by in vitro and in vivo assays. Such assays employ immunization of an animal, e.g., a rabbit or a primate, with the peptide, and evaluation of titers antibody to HIV-1 or to synthetic detector peptides corresponding to variant HIV sequences (see, EXAMPLE 3, and FIG. 10). Methods of determining the spatial conformation of amino acids are known in the art, and include, for example, x-ray crystallography and 2-dimensional nuclear magnetic resonance.

Polynucleotides encoding the peptides. Polynucleotides can encode HIV vaccine candidate peptides, including peptides fused to carrier proteins. HIV vaccine candidate peptides can be encoded by either a synthetic or recombinant polynucleotide. The term “recombinant” refers to the molecular biological technology for combining polynucleotides to produce useful biological products, and to the polynucleotides and peptides produced by this technology. The polynucleotide can be a recombinant construct (such as a vector or plasmid) which contains the polynucleotide encoding the HIV vaccine candidate peptide or fusion protein under the operative control of polynucleotides encoding regulatory elements such as promoters, termination signals, and the like. “Operatively linked” refers to a juxtaposition wherein the components so described are in a relationship permitting them to function in their intended manner. A control sequence operatively linked to a coding sequence is ligated such that expression of the coding sequence is achieved under conditions compatible with the control sequences. “Control sequence” refers to polynucleotide sequences which are necessary to effect the expression of coding and non-coding sequences to which they are ligated. Control sequences generally include promoter, ribosomal binding site, and transcription termination sequence. In addition, “control sequences” refers to sequences which control the processing of the peptide encoded within the coding sequence; these can include, but are not limited to sequences controlling secretion, protease cleavage, and glycosylation of the peptide. The term “control sequences” is intended to include, at a minimum, components whose presence can influence expression, and can also include additional components whose presence is advantageous, for example, leader sequences and fusion partner sequences. A “coding sequence” is a polynucleotide sequence which is transcribed and translated into a polypeptide. Two coding polynucleotides are “operably linked” if the linkage results in a continuously translatable sequence without alteration or interruption of the triplet reading frame. A polynucleotide is operably linked to a gene expression element if the linkage results in the proper function of that gene expression element to result in expression of the HIV vaccine candidate coding sequence. “Transformation” is the insertion of an exogenous polynucleotide (i.e., a “transgene”) into a host cell. The exogenous polynudeotide is integrated within the host genome. A polynucleotide is “capable of expressing” a HIV vaccine candidate peptide if it contains nucleotide sequences which contain transcriptional and translational regulatory information and such sequences are “operably linked” to polynucleotide which encode the HIV vaccine candidate peptide. A polynucleotide that encodes a peptide coding region can be then amplified, for example, by preparation in a bacterial vector, according to conventional methods, for example, described in the standard work Sambrook et al., Molecular Cloning. A Laboratory Manual (Cold Spring Harbor Press 1989). Expression vehicles include plasmids or other vectors. Prokaryotic vectors known in the art include plasmids such as those capable of replication in E. coli (such as, for example, pBR322, ColE1, pSC101, pACYC184, πVX).

The polynucleotide encoding the HIV vaccine candidate peptide can be prepared by chemical synthesis methods or by recombinant techniques. The polypeptides can be prepared conventionally by chemical synthesis techniques, such as described by Merrifield, 85 J. Amer. Chem. Soc. 2149-2154 (1963) (see, Stemmer et al, 164 Gene 49 (1995)). Synthetic genes, the in vitro or in vivo transcription and translation of which will result in the production of the protein can be constructed by techniques well known in the art (see Brown et al., 68 Methods in Enzymology 109-151 (1979)). The coding polynucleotide can be generated using conventional DNA synthesizing apparatus such as the Applied Biosystems Model 380A or 380B DNA synthesizers (commercially available from Applied Biosystems, Inc., 850 Lincoln Center Drive, Foster City, Calif. 94404).

Alternatively, systems for cloning and expressing HIV vaccine candidate peptides include various microorganisms and cells which are well known in recombinant technology. These include, for example, various strains of E. coli, Bacillus, Streptomyces, and Saccharomyces, as well as mammalian, yeast and insect cells. Suitable vectors are known and available from private and public laboratories and depositories and from commercial vendors. See, Sambrook et al., Molecular Cloning. A Laboratory Manual (Cold Spring Harbor Press 1989). See, also PCT International patent application WO 94/01139). These vectors permit infection of patient's cells and expression of the synthetic gene sequence in vivo or expression of it as a peptide or fusion protein in vitro.

Polynucleotide gene expression elements useful for the expression of cDNA encoding peptides include, but are not limited to (a) viral transcription promoters and their enhancer elements, such as the SV40 early promoter, Rous sarcoma virus LTR, and Moloney murine leukemia virus LTR; (b) splice regions and polyadenylation sites such as those derived from the SV40 late region; and (c) polyadenylation sites such as in SV40. Recipient cells capable of expressing the HIV vaccine candidate gene product are then transfected. The transfected recipient cells are cultured under conditions that permit expression of the HIV vaccine candidate gene products, which are recovered from the culture. Host mammalian cells, such as Chinese Hamster ovary cells (CHO) or COS-1 cells, can be used. These hosts can be used in connection with poxvirus vectors, such as vaccinia or swinepox. Suitable non-pathogenic viruses which can be engineered to carry the synthetic gene into the cells of the host include poxviruses, such as vaccinia, adenovirus, retroviruses and the like. A number of such non-pathogenic viruses are commonly used for human gene therapy, and as carrier for other vaccine agents, and are known and selectable by one of skill in the art. The selection of other suitable host cells and methods for transformation, culture, amplification, screening and product production and purification can be performed by one of skill in the art by reference to known techniques (see, e.g., Gething & Sambrook, 293 Nature 620-625 (1981)). Another preferred system includes the baculovirus expression system and vectors.

The general construction and use of polynucleotides encoding for non-infectious, replication-defective, self-assembling HIV-1 viral particles containing HIV antigenic markers is disclosed in U.S. Pat. 5,866,320, the contents of which are incorporated by reference.

The polynucleotide encoding the HIV vaccine candidate peptide can be used in a variety of ways. For example, a polynucleotide can express the HIV vaccine candidate peptide in vitro in a host cell culture. The expressed HIV vaccine candidate peptide immunogens, after suitable purification, can then be incorporated into a pharmaceutical reagent or vaccine (described below).

Alternatively, the polynucleotide encoding the HIV vaccine candidate peptide immunogen can be administered directly into a human as so-called “naked DNA” to express the peptide immunogen in vivo in a patient. (see, Cohen, 259 Science 1691-1692 (1993); Fynan et al., 90 Proc. Natl. Acad. Sci. USA, 11478-82 (1993); and Wolff et al., 11 BioTechniques 474485 (1991). The polynucleotide encoding the HIV vaccine candidate peptide immunogen can be used for direct injection into the host. This results in expression of the HIV vaccine candidate peptide by host cells and subsequent presentation to the immune system to induce anti-HIV antibody formation in vivo.

Determinations of the sequences for the polynucleotide coding region that codes for the HIV vaccine candidate peptides described herein can be performed using commercially available computer programs, such as DNA Strider and Wisconsin GCG. Owing to the natural degeneracy of the genetic code, the skilled artisan will recognize that a sizable yet definite number of DNA sequences can be constructed which encode the claimed peptides (see, Watson et al., Molecular Biology of the Gene, 436437 (the Benjamin/Cummings Publishing Co. 1987)).

Treatment of HIV infection. The method for reducing the viral levels of HIV-1 involves exposing a human to a HIV vaccine candidate peptides, actively inducing antibodies that react with HIV-1, and impairing the multiplication of the virus in vivo. This method is appropriate for an HIV-1 infected subject with a competent immune system, or an uninfected or recently infected subject. The method induces antibodies which react with HIV-1 , which antibodies reduce viral multiplication during any initial acute infection with HIV-1 and minimize chronic viremia leading to AIDS. This method also lowers chronic viral multiplication in infected subjects, minimizing progression to AIDS. In other words, in already infected patients, this method of reduction of viral levels can reduce chronic viremia and progression to AIDS. In uninfected humans, this administration of the peptides of the invention can reduce acute infection and thus minimize chronic viremia leading to progression to AIDS.

The terms “treating,” “treatment,” and the like are used herein to mean obtaining a desired pharmacologic or physiologic effect. The effect can be prophylactic in terms of completely or partially preventing a disorder or sign or symptom thereof, or can be therapeutic in terms of a partial or complete cure for a disorder and/or adverse effect attributable to the disorder. “Treating” as used herein covers any treatment and includes: (a) preventing a disorder from occurring in a subject that can be predisposed to a disorder, but has not yet been diagnosed as having it; (b) inhibiting the disorder, ie., arresting its development; or (c) relieving or ameliorating the disorder, e.g., cause regression of HIV infection or AIDS. An “effective amount” or “therapeutically effective amount” is the amount sufficient to obtain the desired physiological effect, e.g., treatment of HIV. An effective amount of the HIV vaccine candidate peptide or vector expressing HIV vaccine candidate peptides is generally determined by the physician in each case on the basis of factors normally considered by one skilled in the art to determine appropriate dosages, including the age, sex, and weight of the subject to be treated, the condition being treated, and the severity of the medical condition being treated. Among such patients suitable for treatment with this method are HIV-1 infected patients who are immunocompromised by disease and unable to mount a strong immune response. In later stages of HIV infection, the likelihood of generating effective titers of antibodies is less, due to the immune impairment associated with the disease. Also among such patients are HIV-1 infected pregnant women, neonates of infected mothers, and unimmunized patients with putative exposure (e.g., a human who has been inadvertently “stuck” with a needle used by an HIV-1 infected human).

Method of administration HIV vaccine candidate peptides can be administered in a variety of ways, orally, topically, parenterally e.g. subcutaneously, intraperitoneally, by viral infection, intravascularly, etc. Depending upon the manner of introduction, the HIV vaccine candidate peptides can be formulated in a variety of ways. The concentration of HIV vaccine candidate peptides in the formulation can vary from about 0.1-100 wt. %.

The amount of the HIV vaccine candidate peptide or polynucleotides of the invention present in each vaccine dose is selected with regard to consideration of the patients age, weight, sex, general physical condition and the like. The amount of HIV vaccine candidate peptide required to induce an immune response, preferably a protective response, or produce an exogenous effect in the patient without significant adverse side effects varies depending upon the pharmaceutical composition employed and the optional presence of an adjuvant. Generally, for the compositions containing HIV vaccine candidate peptide, each dose will comprise between about 50 μg to about 1 mg of the HIV vaccine candidate peptide immunogens/ml of a sterile solution. A more preferred dosage can be about 200 μg of HIV vaccine candidate peptide immunogen. Other dosage ranges can also be contemplated by one of skill in the art. Initial doses can be optionally followed by repeated boosts, where desirable. The method can involve chronically administering the HIV vaccine candidate peptide composition. For therapeutic use or prophylactic use, repeated dosages of the immunizing compositions can be desirable, such as a yearly booster or a booster at other intervals. The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient can be about 0.01 to 100 mg/kg of body weight. Ordinarily 1.0 to 5, and preferably 1 to 10 mg/kg/day given in divided doses 1 to 6 times a day or in sustained release form is effective to obtain desired results.

The HIV vaccine candidate peptide can be employed in chronic treatments for subjects at risk of acute infection due to needle sticks or maternal infection. A dosage frequency for such “acute” infections may range from daily dosages to once or twice a week i.v. or i.m., for a duration of about 6 weeks. The peptides can also be employed in chronic treatments for infected patients, or patients with advanced HIV. In infected patients, the frequency of chronic administration can range from daily dosages to once or twice a week i.v. or i.m., and may depend upon the half-life of the immunogen (e.g., about 7-21 days). However, the duration of chronic treatment for such infected patients is anticipated to be an indefinite, but prolonged period.

For such therapeutic uses, the HIV vaccine candidate peptide formulations and modes of administration are substantially identical to those described specifically above and can be administered concurrently or simultaneously with other conventional therapeutics for the viral infection.

Immunologically acceptable carrier. HIV vaccine candidate peptides can be administered either as individual therapeutic agents or in combination with other therapeutic agents. HIV vaccine candidate peptides can be administered alone, but are generally administered with a pharmaceutical carrier selected on the basis of the chosen route of administration and standard pharmaceutical practice. The vaccine can further comprise suitable, i.e., physiologically acceptable, carriers—preferably for the preparation of injection solutions—and further additives as usually applied in the art (stabilizers, preservatives, etc.), as well as additional drugs. The patients can be administered a dose of approximately 1 to 10 μg/kg body weight, preferably by intravenous injection once a day. For less threatening cases or long-lasting therapies the dose can be lowered to 0.5 to 5 μg/kg body weight per day. The treatment can be repeated in periodic intervals, e.g., two to three times per day, or in daily or weekly intervals, depending on the status of HIV-1 infection or the estimated threat of an individual of getting HIV infected.

For parenteral administration, peptides of the invention can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by commonly used techniques. Suitable pharmaceutical carriers are described in the most recent edition of Remington's Pharmaceutical Sciences, a standard reference text in this field of art. For example, a parenteral composition suitable for administration by injection is prepared by dissolving 1.5% by weight of active ingredient in 0.9% sodium chloride solution. The preparation of these pharmaceutically acceptable compositions, having appropriate pH isotonicity, stability and other conventional characteristics is within the skill of the art.

The vaccine composition can include as the active agents, one of the following above-described components: (a) a HIV vaccine candidate peptide immunogen (These immunogens can be in the form of recombinant proteins. Alternatively, they can be in the form of a mixture of carrier protein conjugates.); (b) a polynucleotide encoding a HIV vaccine candidate; (c) a recombinant virus carrying the synthetic gene or molecule; and (d) a bacteria carrying the HIV vaccine candidate. The selected active component is present in a pharmaceutically acceptable carrier, and the composition can contain additional ingredients.

Formulations containing the HIV vaccine candidate peptide can contain other active agents, such as adjuvants and immunostimulatory cytokines, such as IL-12 and other well-known cytokines, for the peptide compositions.

Suitable pharmaceutically acceptable carriers for use in an immunogenic composition are well known to those of skill in the art. Such carriers include, for example, saline, a selected adjuvant, such as aqueous suspensions of aluminum and magnesium hydroxides, liposomes, oil in water emulsions, and others.

Carrier protein. HIV vaccine candidate peptide immunogens can be linked to a suitable carrier in order to improve the efficacy of antigen presentation to the immune system. Such carriers can be, for instance, organic polymers. A carrier protein can enhance the immunogenicity of the peptide immunogen. Such a carrier can be a larger molecule which has an adjuvant effect. Exemplary conventional protein carriers include, keyhole limpet hemocyan, E. coli DnaK protein, galactokinase (galK, which catalyzes the first step of galactose metabolism in bacteria), ubiquitin, α-mating factor, β-galactosidase, and influenza NS-1 protein. Toxoids ( i.e., the sequence which encodes the naturally occurring toxin, with sufficient modifications to eliminate its toxic activity) such as diphtheria toxoid and tetanus toxoid can also be employed as carriers. Similarly a variety of bacterial heat shock proteins, e.g., mycobacterial hsp-70 can be used. Glutathione reductase (GST) is another useful carrier. One of skill in the art can readily select an appropriate carrier.

Viruses can be modified by recombinant DNA technology such as, e.g. rhinovirus, poliovirus, vaccinia, or influenzavirus, etc. The peptide can be linked to a modified, i.e., attenuated or recombinant virus such as modified influenza virus or modified hepatitis B virus or to parts of a virus, e.g., to a viral glycoprotein such as, e.g., hemagglutinin of influenza virus or surface antigen of hepatitis B virus, in order to increase the immunological response against HIV-1 viruses and/or infected cells.

The HIV vaccine candidate peptides can be in fusion proteins, wherein they are linked to a suitable carrier which might be a recombinant or attenuated virus or a part of a virus such as, e.g., the hemagglutinin of influenza virus or the surface antigen of hepatitis B virus, or another suitable carrier including other viral surface proteins, e.g., surface proteins of rhinovirus, poliovirus, sindbis virus, coxsackievirus, etc., for efficient presentation of the antigenic site(s) to the immune system. In some cases, the antigenic fragments might, however, also be purely, i.e., without attachment to a carrier, applied in an analytical or therapeutical program.

Naked DNA vaccine. Alternatively, polynucleotides can be designed for direct administration as “naked DNA”. Suitable vehicles for direct DNA, plasmid polynucleotide, or recombinant vector administration include, without limitation, saline, or sucrose, protamine, polybrene, polylysine, polycations, proteins, calcium phosphate, or spermidine. See e.g, PCT International patent application WO 94/01139. As with the immunogenic compositions, the amounts of components in the DNA and vector compositions and the mode of administration, e.g., injection or intranasal, can be selected and adjusted by one of skill in the art. Generally, each dose will comprise between about 50 μg to about 1 mg of immunogen-encoding DNA per ml of a sterile solution.

For recombinant viruses containing the coding polynucleotide, the doses can range from about 20 to about 50 ml of saline solution containing concentrations of from about 1×10⁷ to 1×10¹⁰ pfu/ml recombinant virus of the invention. One human dosage is about 20 ml saline solution at the above concentrations. However, it is understood that one of skill in the art can alter such dosages depending upon the identity of the recombinant virus and the make-up of the immunogen that it is delivering to the host.

The amounts of the commensal bacteria carrying the synthetic gene or molecules to be delivered to the patient will generally range between about 10³ to about 10¹² cells/kg. These dosages, will of course, be altered by one of skill in the art depending upon the bacterium being used and the particular composition containing immunogens being delivered by the live bacterium.

Antibodies. An antibody directed against a HIV vaccine candidate peptide is also an aspect of this invention. Polyclonal antibodies are produced by immunizing a mammal with a peptide immunogen. Suitable mammals include primates, such as monkeys; smaller laboratory animals, such as rabbits and mice, as well as larger animals, such as horse, sheep, and cows. Such antibodies can also be produced in transgenic animals. However, a desirable host for raising polyclonal antibodies to a composition of this invention includes humans. The polyclonal antibodies raised are isolated and purified from the plasma or serum of the immunized mammal by conventional techniques. Conventional harvesting techniques can include plasmapheresis, among others. Such polyclonal antibodies can themselves be employed as pharmaceutical compositions of this invention. Alternatively, other forms of antibodies can be developed using conventional techniques, including monoclonal antibodies, chimeric antibodies, humanized antibodies and fully human antibodies (see, e.g., U.S. Pat. No. 4,376,110; Ausubel et al., Current Protocols in Molecular Biology (Greene Publishing Assoc. and Wiley Interscience, N.Y., 1992); Harlow & Lane, Antibodies: a Laboratory Manual, (Cold Spring Harbor Laboratory, 1988); Queen et al., 86 Proc. Nat'l. Acad. Sci. USA 10029-10032 (1989); Hodgson et al., 9 Bio/Technology 421 (1991); PCT International patent application WO 92/04381 and PCT International patent application WO 93/20210. Other antibodies can be developed by screening hybridomas or combinatorial libraries, or antibody phage displays (Huse et al., 246 Science 1275-1281 (1988) using the polyclonal or monoclonal antibodies produced according to this invention and the amino acid sequences of the primary or optional immunogens.

The term “antibody” includes polyclonal antibodies, monoclonal antibodies (mAbs), chimeric antibodies, anti-idiotypic (anti-Id) antibodies to antibodies that can be labeled in soluble or bound form, as well as fragments, regions or derivatives thereof, provided by any known technique, such as, but not limited to enzymatic cleavage, peptide synthesis or recombinant techniques. An “antigen binding region” is that portion of an antibody molecule which contains the amino acid residues that interact with an antigen and confer on the antibody its specificity and affinity for the antigen. The antibody region includes the framework amino acid residues necessary to maintain the proper conformation of the antigen-binding residues.

Computer Implementation. Aspects of the invention may be implemented in hardware or software, or a combination of both. However, preferably, the algorithms and processes of the invention are implemented in one or more computer programs executing on programmable computers each comprising at least one processor, at least one data storage system (including volatile and non-volatile memory and/or storage elements), at least one input device, and at least one output device. Program code is applied to input data to perform the functions described herein and generate output information. The output information is applied to one or more output devices, in known fashion.

Each program may be implemented in any desired computer language (including machine, assembly, high level procedural, or object oriented programming languages) to communicate with a computer system. In any case, the language may be a compiled or interpreted language.

Each such computer program is preferably stored on a storage media or device (e.g., ROM, CD-ROM, tape, or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

The details of one or more embodiments of the invention are set forth in the accompanying description. Although any methods and materials similar or equivalent to those described herein can be used in the practice or testing of the invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The following EXAMPLES are presented in order to more fully illustrate the preferred embodiments of the invention. These examples should in no way be construed as limiting the scope of the invention, as defined by the appended claims.

### EXAMPLE 1

### Prediction of Well-conserved HIV-1 Ligands using a Matrix-based Algorithm, Epimatrix

Summary. This EXAMPLE was undertaken to identify new human leukocyte antigens (HLA) ligands from human immunodeficiency virus type 1 (HIV-1) which are highly conserved across HIV-1 clades and which may serve to induce cross-reactive cytotoxic T lymphocytes (CTLs). EpiMatrix was used to predict putative ligands from HIV-1 for HLA-A2 and HLA-B27. Twenty-six peptides that were both likely to bind and also highly conserved across HIV-1 strains in the Los Alamos HIV sequence database were selected for binding assays using the T2 stabilization assay. Two peptides that were also highly likely to bind (for A2 and B27, as determined by EpiMatrix) and well conserved across HIV-1 strains, and had previously been described to bind in the publicized literature, were also selected to serve as positive controls for the assays. Ten new major histocompatibility complex (MHC) ligands were identified among the 26 study peptides. The control peptides bound, as expected. These data confirm that EpiMatrix can be used to screen HIV-1 protein sequences for highly conserved regions that are likely to bind to MHC and may prove to be highly conserved HIV-1 CTL epitopes.

Introduction. This EXAMPLE is a prospective design of multivalent HIV immunogens tailored to reflect the diversity of HIV isolates and to promote cross-clade protection in settings where more than one HIV strain and more than one HIV clade is being transmitted. This EXAMPLE explored the use of EpiMatrix, a matrix-based algorithm for T-cell epitope prediction, to prospectively identify conserved class I-restricted MHC ligands and potential CTL epitopes. EpiMatrix and other computer-driven algorithms that predict putative MHC ligands and CTL epitopes (Davenport et al., 42 Immunogenetics 392-7 (1995); Hammer et al., 180 J. Exp. Med. 2353-8 (1994); Flackenstein et al., 240 Eur. J. Biochem. 71-7 (1996)) place the prospective design of a novel HIV-1 vaccine with these critically important characteristics within reach.

Such prospectively designed vaccines are based on the central role of CTL in the host immune response to HIV-1, and the understanding that the first step in the search for HIV-1 CTL epitopes may be to identify peptides that bind to the host major histocompatibility complex (MHC). Recognition of such MHC ligands by CTL is dependent on the presentation of the T-cell epitope to the T cells in the context of MHC molecules. Peptides presented in conjunction with class I MHC molecules (to T cells) are derived from foreign or self-protein antigens that have been processed in the cytoplasm. The peptides bind to MHC molecules in a linear fashion; the binding is determined by the interaction of the peptide's amino acid side-chains with binding pockets in the MHC molecule. Binding of peptides to MHC molecules is constrained by the nature of the side-chains; only selected peptides will fit the constraints of any given MHC molecule's binding pockets.

The characteristics of peptides that are likely to bind to a given MHC can be directly deduced from pooled sequencing data (from peptides bulk-eluted off MHC molecules), from MHC binding peptide libraries. The TB/HIV Research Lab has developed a method to describe the relative promotion or inhibition of binding afforded by each position in a peptide to the MHC of interest.

EpiMatrix ranks all 10 amino acid long segments from any protein sequence by estimated probability of binding to a given MHC, by comparing the sequence to a matrix. The estimated binding probability (EBP) is derived by comparing the EpiMatrix score to those of known binders and presumed non-binders. Retrospective studies have demonstrated that EpiMatrix accurately predicts MHC Ligands (DeGroot et al., 7 Human Retroviruses 139 (1997); Jesdale et al., in Vaccines '97. (Cold Spring Harbor Press, Cold Spring Harbor, 1997).

In this EXAMPLE, we implemented EpiMatrix to examine the sequences of HIV-1 strains published on the 1995 version of the Los Alamos National Laboratory HIV Sequence database. We identified conserved regions and then examined these for their potential to bind to one of two MHC alleles (A2 and B27). We prospectively identified conserved MHC ligands which may be useful for HIV-1 vaccine development.

Generation of an MHC binding matrix motif. Various methods were used in the generation of MHC binding matrix motifs. Briefly, independent sources of information on the relative promotion or inhibition of each amino acid in each position are identified. For each source of information, an estimation of the relative promotion or inhibition of binding is quantified. In a generic sense, this quantification is based on a relative rate calculation, the rate of an amino acid in a given position relative to its median rate across all positions. These matrix motifs, based on single sources of information (such as a list of known ligands (Huczko et al., 151 J. Immunol. 2572 (1993)); pooled sequencing of naturally elated peptides (Kubo et al., 152 J. Immunol. 3913-24 (1993)) peptide side-chain scanning techniques (Hammer et al., 180 J. Exp. Med. 2353-8 (1994)), or the identification of ligands with specific characteristics through random phage techniques Flackenstein et al., 240 Eur. J. Biochem. 71-7 (1996)), are then combined in a way which attempts to maximize the resultant matrix motifs ability to separate a list of known ligands from the other peptides contained within their original sequences. The two matrix motifs based on single datasets with the best individual predictive power (assessed using the Kruskal—Wallis non-parametric test) are first combined with each other. The best resultant of these two was then combined with the third most individually predictive, and so on. The result of this process was then combined with the method of Parker et al., 152 J. Immunol. 163-75 (1994) to achieve a final predictive matrix motif for each MHC allele.

Generating an EpiMatrix score. Each putative MHC binding region within a given protein sequence is scored by estimating the relative promotion or inhibition of binding for each amino acid, and summing these to create a summary score for the entire peptide. Higher EpiMatrix scores indicate greater MHC binding potential. After comparing the score to the scores of known MHC ligands, an “estimated binding probability” or EBP, is estimated. The EBP describes the proportion of peptides with EpiMatrix scores as high or higher that will bind to a given MHC molecule.

EBP is derived from the EpiMatrix score by determining how many published ligands for the allele would earn that same score or a higher score (a measure of sensitivity). EBPs range from 100% (highly likely to bind) to less than 1% (very unlikely to bind). The majority of 10mers in any one protein sequence fall below the 1% estimated binding probability for any given MHC binding matrix.

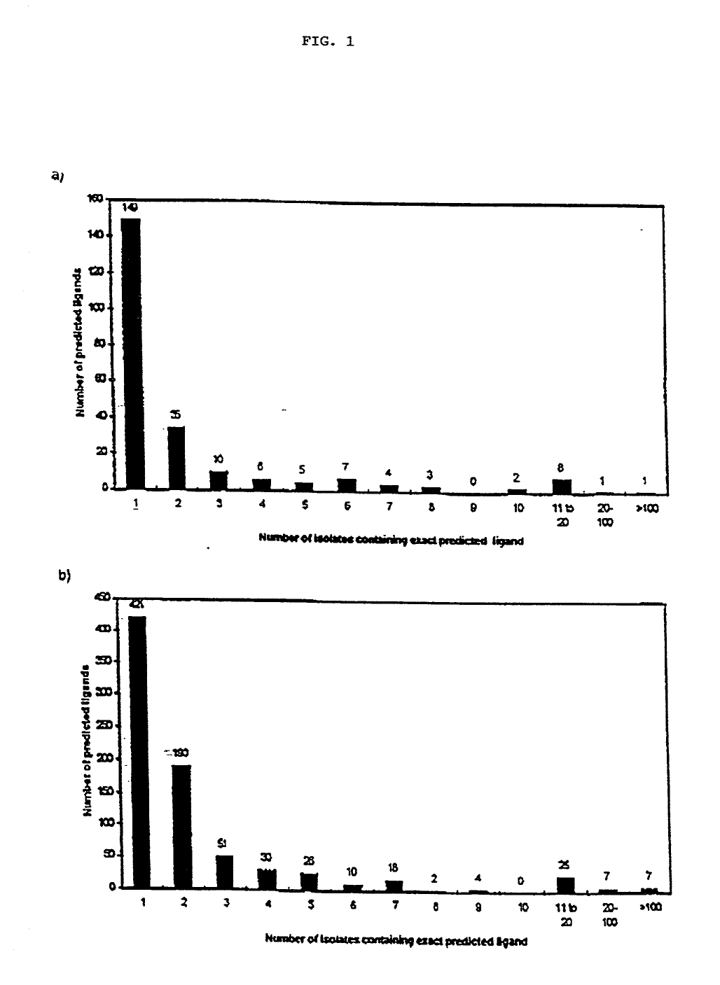

Selection of peptides. For each protein, env, pol, nef, and tat was analyzed independently. The sequence for each HIV-1 isolate in the Los Alamos HIV sequence database (Korber & Meyers, eds, HIV Sequence Database, Los Alamos HIV Database, 1995. (Los Alamos National Laboratories, New Mexico, 1995) was divided into ten amino acid long strings which overlapped by nine. These 10-mer strings were then compared to the A2 and B7 MHC binding matrix motifs (EpiMatrix version 1.0). Peptides that scored higher than 50% EBP were selected. Each of these putative ligands was compared to all the others using a spreadsheet and command macro which orders the strings from those which are common to many of the sequences to those which were unique (FIG. 1). Strings that were present in “more” HIV-1 isolates (the exact number depended on the number of isolates available in the LANL database) were selected for the next phase of the analysis. Twenty-eight peptides were selected using this method. One of the selected peptides corresponded to a published CTL epitope, and was selected to serve as a control. An additional peptide selected to serve as a positive control as for this study, KRWIILGLNK, scored lower on the B27 matrix than 50%, however, it was the only available HIV-1 B27 ligand that had been fine-mapped.

The T2 in vitro peptide binding assay was performed as recently described by Nijman et al., 23 Eur. J. Immunol. 1215-9 (1993). This assay relies on the ability of exogenously added peptides to stabilize the Class I/β2 microglobulin structure on the surface of TAP-defective cell lines. For these assays, we used the antigen processing mutant cell line T2 transfected with the HLA B27 gene (T2/B27). These cells were cultured in Iscove Modified Dulbecco's Medium (IBM), 10% fetal bovine serum, and 20 μg/ml gentamycin. A monoclonal antibody to HLA-827 produced by the ATCC 1-HB-119. MEI hybridoma (Ellis et al., 5 Hum. Immunol. 49-59 (1982) was used to assess HLA-B27 expression at the cell surface (indicating peptide binding and stabilization of the B27 molecule). The monoclonal antibody produced by the ATCC HB-82, BB7.2 hybridoma (Parham & Brodsky, 3 Hum. Immunol. 277-99 (1981)) was used to assess HLA-A2 expression at the cell surface.

Three hundred thousand cells in 100 μl of IMDM, 10% FBS, and 20 μg/ml gentamycin medium were incubated with no peptide, or 100 μl synthetic peptide solution overnight at 37° C., in an atmosphere of 5% CO₂. The T2 cell/peptide suspension was pelleted at 1000 rpm. the supernatant was discarded, and the suspension was stained with 100 μl of BB7.2, an HLA-A2 specific mouse monoclonal primary antibody (1 hr at 4° C.). Two wells per peptide did not receive the primary antibody, but only the PBS staining buffer. The cells were washed 3× with cold (4° C.) staining butter PBS, 0.5% FBS, 0.02% NaN₃, and stained for 30 min at 4° C. with 100 ill FITC-labeled goat anti-mouse immunoglobulin (Pharmingen, 12064-D). The cells were again washed three times and fixed in 1% paraformaldehyde. Fluorescence of viable T2 cells was measured at 488 nm on a FACScan flow cytometer (Becton-Dickinson, NJ).

A total of 12 wells was assayed per peptide (one well each with peptide at 0, 2, 20, and 200 μg/ml were repeated using primary antibody for the molecule the peptide is predicted to bind to, the primary antibody to the molecule the peptide was not predicted to bind to, and no primary antibody).

Analysis and interpretation of binding assays. Peptide binding to MHC molecules stabilizes MHC expression at the cell surface, and can be measured by FACS sorting the cells. The data produced by the FACS analysis represented the mean linear fluorescence (MLF) of 10000 events. We used a cut-off of 1.3-fold greater MFI in any of the three wells with peptide than the control well as the criterion for positive binding.

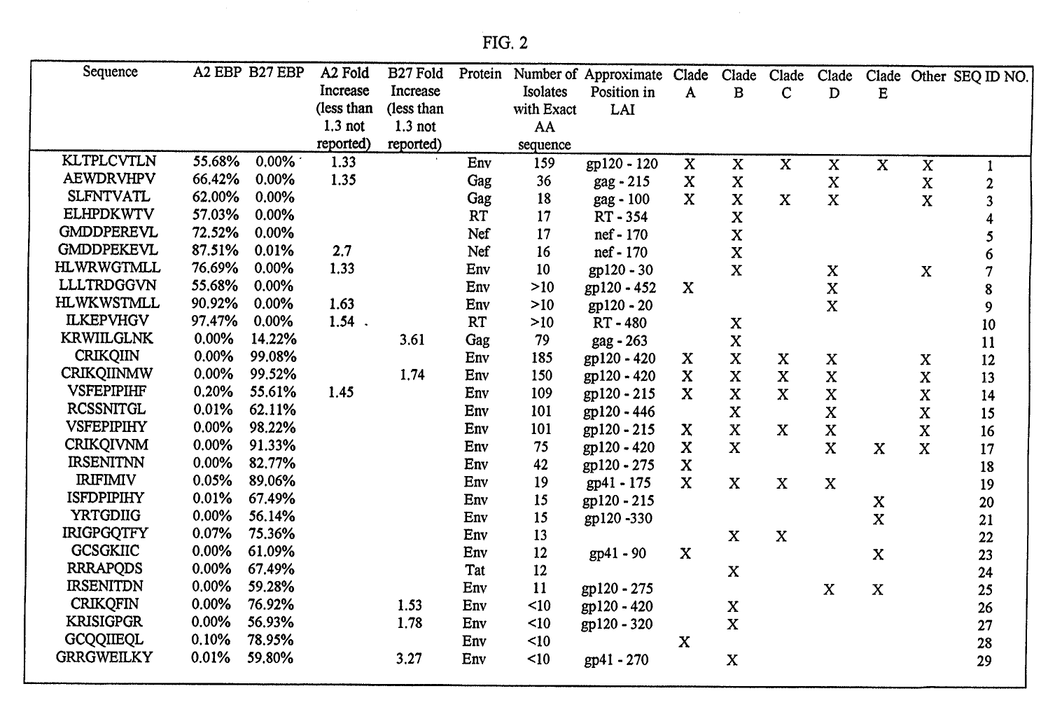

Results. Twenty-eight peptides were tested in binding assays. Two of the 28 were previously published ligands. Ten peptides induced an increase in the MFI of 1.3-fold or greater (FIG. 2). The published controls bound as expected. Peptides shown here were selected because they were predicted to bind to A2 and not to B27, or vice versa. None of the peptides predicted to bind to A2 bound to B27 and vice versa.

Conclusion We performed prospective definition of conserved HIV-1 regions using EpiMatrix version 1.0. Rapid identification of MHC ligands, which can then be tested in T-cell assays, is desirable for HIV-1 vaccine development. Computer-driven analysis of HIV sequences will permit the prospective identification of such conserved CTL epitopes.

Determination of peptides that bind to major history compatibility (MHC) molecules (MHC ligands) can be the first step in the process of identifying T-cell epitopes. Identification of MHC ligands from primary HIV-1 sequences as particularly relevant for HIV vaccine development and immunopathogenesis research. Matrix-based motifs have been developed to improve on the specificity of anchor-based motifs. The advantage of matrix motifs is that peptides can be given a score that represents the sum of the potential for each ammo acid in the sequence to promote or inhibit binding.

Predicting regions of immunological interest is only the first step to determining whether the region is likely to be recognized by primed T cells, and to be defined as a CTL epitope. Predictions must be confirmed by binding assays, so as to determine whether a peptide representing that region indeed binds to the MHC for which it was predicted (e.g., T2 cell binding assay). Immunogencity of the peptides must also be confirmed by measuring whether CTL recognize the peptide in T-cell assays.

Methods of analysis developed in the TB/HIV Research Lab also permit the comparison of putative MEC ligands across HIV-1 clades and permit the weighting of predictions for the prevalence of HLA alleles in human populations. Utilization of these computer-driven methods will put the prospective identification of cross-clade (cross-reactive) and promiscuous epitopes for HIV-1 vaccine development within reach.

### EXAMPLE 2

### A Regional HIV Vaccine for India

Introduction. India has one of the highest burdens of HIV infection of any country in the world: 4.1 million individuals are already thought to be infected and the epidemic will accelerate over the next decade. The prevalence of selected clades on the Indian sub-continent and the unique genetic make-up (HLA distribution) of the Indian population led to the concept of a region-specific HIV vaccine.

We selected HIV peptides for conservation across HIV-1 strains that have been isolated in India. We then evaluated these peptides for their projected binding capability to selected MHC Class I molecules, using the computer-driven modeling program, EpiMatrix. Twenty eight peptides were identified as highly conserved in the Indian HIV-1 sequences and predicted to bind to MHC Class I (HLA-A0201, -A1101, -B35, -B7) that are prevalent HLA alleles in India.

Analysis. Sixty six HIV-1 sequences from India (55 env, 6 gag, 5 pol) were identified from published literature as having been isolated in India or from individuals who acquired their HIV infection in India. The amino acid sequences were examined for regions conserved in ˜50% of the sequences. These peptides were synthesized and tested in vitro using an MHC binding assay protocol. CTL assays were also performed. Fluorescence data was analyzed using: (1) a two-factor ANOVA to determine treatment or plate effect, and (2) a multiple comparison to find significant differences between treatment means.

Results. Twenty out of the 28 predicted peptides (71%) stabilized the MHC Class I molecule for which they were predicted to bind. (p-values <0.001). The predictive accuracy of the B7 (86%) and B35 (100%) matrices for the EpiMatrix algorithm were slightly better, in this EXAMPLE, than the accuracies of the A11(42%) and A2(57%) matrices. B7 peptides predicted to bind to B35 as well were able to stabilize B35 in vitro. B7 Peptides predicted to be unlikely to bind to B35 did not stabilize B35 in vitro. The reverse (B35/B7) was also true.

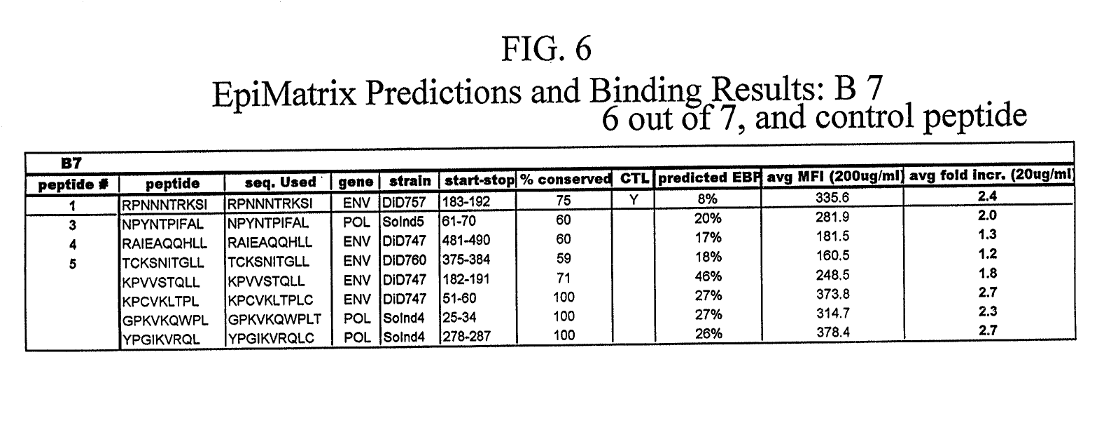

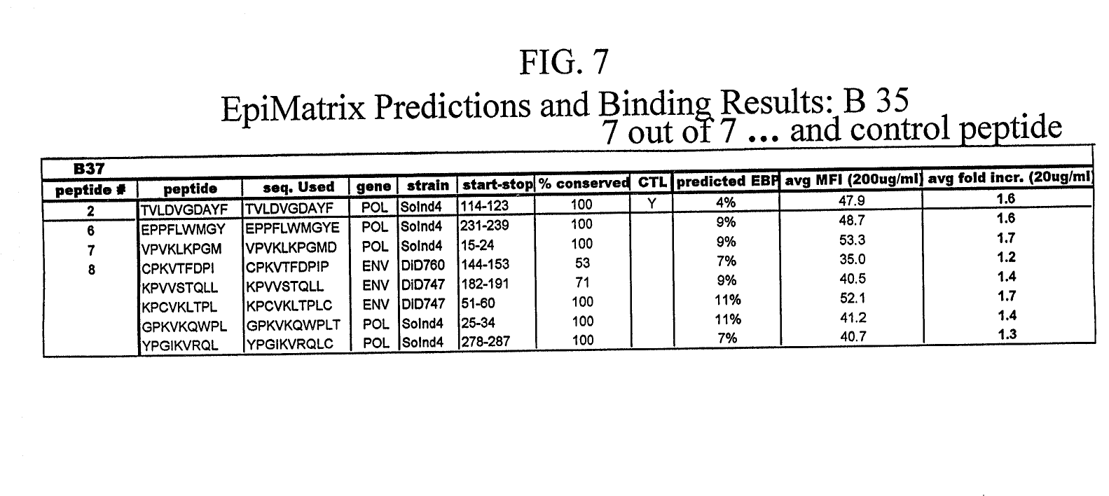

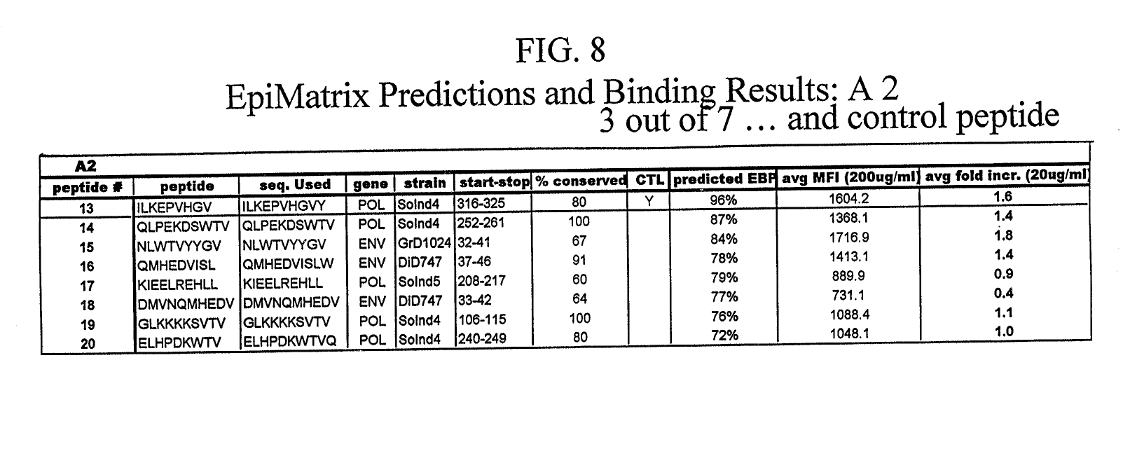

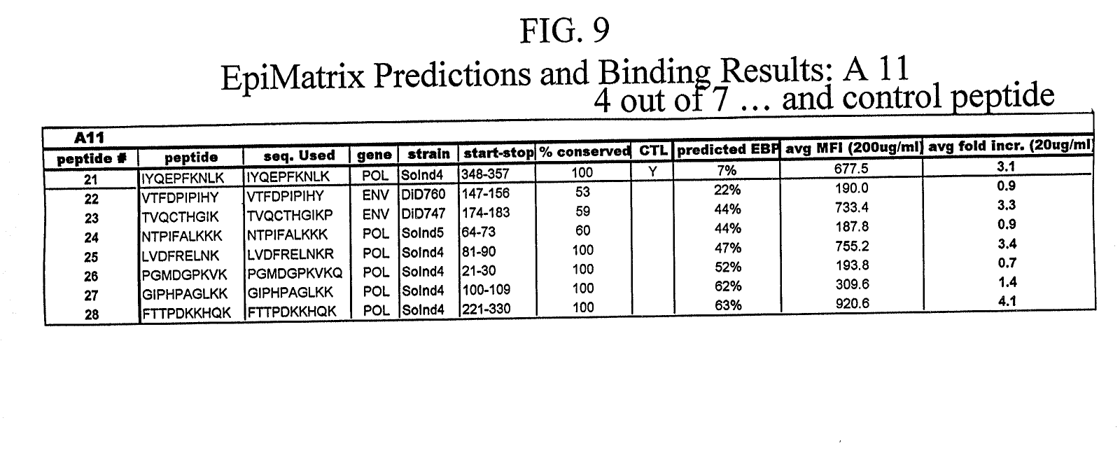

The following TABLES correspond to FIGS. 6-9.

TABLE 1
B7

| peptide # | peptide | seq. Used | SEQ ID NO: |
|---|---|---|---|
| 1 | RPNNNTRKSI | RPNNNTRKSI | 627 |
| 3 | NPYNTPIFAL | NPYNTPIFAL | 628 |
| 4 | RAIEAQQHLL | RAIEAQQHLL | 629 |
| 5 | TCKSNITGLL | TCKSNITGLL | 630 |
| 9 | KPVVSTQLL | KPVVSTQLL | 631 |
| 10 | KPCVKLTPL | KPCVKLTPLC | 632, 633 |
| 11 | GPKVKQWPL | GPKVKQWPLT | 634, 635 |
| 12 | YPGIKVRQL | YPGIKVRQLC | 636, 637 |

TABLE 2
B37

| peptide # | peptide | seq. Used | SEQ ID NO: |
|---|---|---|---|
| 2 | TVLDVGDAYF | TVLDVGDAYF | 638 |
| 6 | EPPFLWMGY | EPPFLWMGYE | 639, 640 |
| 7 | VPVKLKPGM | VPVKLKPGMD | 641, 642 |
| 8 | CPKVTIFDPI | CPKVTFDPIP | 643, 644 |
| 9 | KPVVSTQLL | KPVVSTQLL | 645 |
| 10 | KPCVKLTPL | KPCVKLTPLC | 646, 647 |
| 11 | GPKVKQWPL | GPKVKQWPLT | 648, 649 |
| 12 | YPGIKVRQL | YPGLKVRQLC | 650, 651 |

TABLE 3
A2

| peptide # | peptide | seq. Used | SEQ ID NO: |
|---|---|---|---|
| 13 | ILKEPVHGV | ILKEPVHGVY | 652, 653 |
| 14 | QLPEKDSWTV | QLPEKDSWTV | 654 |
| 15 | NLWTVYYGV | NLWTVYYGV | 655 |
| 16 | QMHEDVISL | QMHEDVISLW | 656, 657 |
| 17 | KIEELREHLL | KIEELREHLL | 658 |
| 18 | DMVNQMHEDV | DMVNQMHEDV | 659 |
| 19 | GLKKKKSVTV | GLKKKKSVTV | 660 |
| 20 | ELHPDKWTV | ELHPDKWTVQ | 661 |

TABLE 4
A11

| peptide # | peptide | seq. Used | SEQ ID NO: |
|---|---|---|---|
| 21 | IYQEPFKNLK | IYQEPFKNLK | 662 |
| 22 | VTFDPIPIHY | VTFDPIPIHY | 663 |
| 23 | TVQCTHGIK | TVQCTHGIKP | 664, 665 |
| 24 | NTPIFALKKK | NTPIFALKKK | 666 |
| 25 | LVDFRELNK | LVDFRELNKR | 667, 668 |
| 26 | PGMDGPKVK | PGMDGPKVKQ | 669,670 |
| 27 | GIPHPAGLKK | GIPHPAGLKK | 671 |
| 28 | FTTPDKKHQK | FTTPDKKHQK | 672 |

Conclusion. Regionalized CTL epitopes can be incorporated into a range of existing vaccine strategies, e.g. vectored vaccines, DNA vaccines, and recombinant protein vaccines. This approach also permit the development of novel regionalized HIV vaccines and therapeutic interventions. Alternatively, such regional CTL epitopes, collectively covering virtually all regionally-transmitted strains and prevalent HLA types could be combined into a universal HIV vaccine.

### EXAMPLE 3

### A “World Clade” HIV Vaccine

HLA Variation in Populations. The distribution of MHC alleles varies from population to population. In general, the MHC-peptide (epitope) interaction is governed by the sequence of the peptide: each MHC has its own constraints, which can be described as a pattern, or motif, characterizing the set of peptides that can bind in the binding groove of the MHC. While the distribution of MHC in populations inhabiting different regions of the world may restrict, to some extent, the relevance of selected epitopes in different human populations, means to surmount this difficulty have been proposed. For example, identification of CTL epitopes that may be recognized in the context of more than one MHC, such as “promiscuous” or “clustered” MHC binding regions, may permit the development of vaccines that effectively protect genetically diverse human populations. For example, if an HIV-1 peptide could be identified that would bind and be presented by A2, A1, and A20, it is likely that it would be presented in the context of MHC of approximately 25% of Zaireans (Congolese) and greater than 50% of North American Caucasians. We and others have proposed that prospectively identifying and including such “promiscuous” CTL and Th epitopes in novel HIV-1 vaccines may enhance the utility of these vaccines in a wide range of HIV-1 endemic countries (Haynes, 348 Lancet 933-937 (1996); Cease & Berzofsky, 12 Annu. Rev. Immunol. 923-989 (1994); Bona et al., 126(19) Immunology Today 126-130 (1998); Brander & Walker, in HIV Immunology Database 1995, Korber & Meyers, eds. (Los Alamos National Laboratories, New Mexico, 1996); Berzofsky et al., 88(3) J. Clin. Invest. 876-84 (1991); Ward et al., in HIV Immunology Database 1995, Korber & Meyers, eds. (Los Alamos National Laboratories, New Mexico, 1996)).

Database of Conserved HIV-1 MHC Ligands. We have prospectively identified regions that are conserved across the maximum number of strains (“cross-clade”) of MHC binding potential that are likely to be presented by MHC molecules representing the most prevalent HLA alleles (“promiscuous”), and has selected, or weighted, the selection of potential CTL epitopes for the final vaccine construct such that HLA alleles prevalent in HIV-endemic regions of the world are adequately represented.

These are highly conserved, promiscuous peptides. Eighty peptides have been synthesized, and binding studies have been intitiated for peptides representing the following alleles: A2, A11, B35, and B7. Studies of peptides representing the following alleles: A1, A3, A24, A31, A33, B12 (44), B17, B53, Cw3, and Cw4 are next in order of priority.

Research Lab Tools; EpiMatrix. EpiMatrix is a matrix-based algorithm that ranks 10 amino acid long segments, overlapping by 9 amino acids, from any protein sequence by estimated probability of binding to a selected MHC molecule. The procedure for developing matrix motifs was published by Schafer et al, 16 Vaccine 1998 (1998). We have constructed matrix motifs for 32 HLA class I alleles, one murine allele (H-2 Kd) and several human class II alleles. Putative MHC ligands are selected by scoring each 10-mer frame in a protein sequence. This score, or estimated binding probability (EBP), is derived by comparing the sequence of the 10-mer to the matrix of 10 amino acid sequences known to bind to each MHC allele. Retrospective studies have demonstrated that EpiMatrix accurately predicts published MHC ligands (Jesdale et al., in Vaccines '97 (Cold Spring Harbor Press, Cold Spring Harbor, N.Y., 1997)).

An additional feature of EpiMatrix is that it can measure the MHC binding potential of each 10 amino acid long snapshot to a number of human HLA, and therefore can be used to identify regions of MHC binding potential clustering. Other laboratories have confirmed cross-presentation of peptides within HLA “superfamilies” (A11, A3, A31, A33 and A68) (Jesdale et al., in Vaccines '97 (Cold Spring Harbor Press, Cold Spring Harbor, N.Y., 1997)). Presumably, vaccines containing such “clustered” or promiscuous epitopes will have an advantage over vaccines composed of epitopes that are not “clustered. In work performed in the TB/HIV Research Lab, we have confirmed cross-MHC binding that was predicted by EpiMatrix.

Peptides Selected for Conservation Across Clades and for CTL Response. The staff of the Los Alamos National Laboratory HIV-1 Sequence Database has compiled a list of HIV-1 sequences which are believed to be representative of currently available HIV-1 sequences. Such representative lists are available for each of the HIV genes/proteins (gag, pol, gag, vpu, env, nef, vif, vpr), although the more heavily sequenced genes (particularly env) have considerably longer lists. It is from these lists that well-conserved putative ligands have been defined.

The list for each protein was analyzed independently. We used a program called Conservatrix, developed in the TB/HIV Research Laboratory, to find conserved regions. The sequence for each isolate was divided into ten amino acid-long strings that overlapped by nine. Each of these strings was compared to all of the others using a spreadsheet program that orders the strings from those which were in many of the sequences to those which were unique (Conservatrix). These ordered lists represent the first step in the analysis. Strings that were present in “more” (>50 for env, >25 for gag, etc.) HIV-1 isolates were selected for the next phase of the analysis. For example, in the case of env, 478 strings were conserved in more than 50 HIV-1 isolates and were analyzed, using EpiMatrix, for MHC binding potential clustering.

The next step was to identify which of the conserved sequences were likely to be MHC ligands (and putatively, CTL epitopes). EpiMatrix yields a “score” for each of the strings it analyzes. The somewhat arbitrary score of 20% estimated binding probability (EBP) was defined as the cut-off for this step in the analysis. This cut-off is probably too high (too specific, not sensitive enough). The complete list of conserved sequences has been archived.

To continue using env as an example, of the 478 conserved env strings, any peptide with an EBP of greater than 20% for any of the HLA for which EpiMatrix predictions were available was defined as being a putative ligand. 206 of the 478 well conserved strings (43%) met this criterion.

The next step was to select strings that were likely to be ligands for more than one MHC type (MHC binding potential clustering). Histograms have been constructed which indicate which regions stimulate the most HLA types (see, TABLE 5 below).

The list of peptides to be tested has been selected from among those regions that might bind to more than 3 different MHC molecules, paying particular attention to selecting regions that bind to HLA representative of world populations and sequences that were representative of global HIV-1 isolates. A method for weighting predictions by the prevalence of HLA alleles in populations has already been developed in the laboratory. We have performed the first two steps of the peptide selection analysis for env, pol, and gag. Twenty-eight of the peptides selected in this manner are shown in TABLE 5 below, with an abbreviated listing of the strains for which they were identified. Binding studies were also performed.

Reviewing the data shown below, it is clear that we have been able to select from a number of different peptides that are conserved in a wide range of HIV-1 clades and strains. The listing of strains for which each peptide is conserved is limited by space for this application; however, it is should be apparent that there is good cross-clade coverage of different HIV-1 clades.

The following TABLE 5 provides a sample list of peptides that are conserved across HIV-1 clades (only env is shown).

| | conserved in | | | number | |
|---|---|---|---|---|---|
| | # of HIV-t | reference | | predicted | Putative ligands for these |
| protein | strains | strain | strains for which sequence is conserved (partial listing) | >20% | alleles |
| env | 70 | SF1703 | Z321 [318] 92UG037.8 [317] TZ017 [310] L414 [55] C1211 [50] UG273A [321] DJ264A [313] DJ263A [317] DJ | 3 | A′6801, B′39011, B′5801 |
| env | 69 | SF2 | LAl [705] KXB2R [700] NL43 [698] BRVA [696] 91US005.11 [706] MN [701] OZ4589 [703] JFL [695] SHR84 [7 | 3 | A′3302, A′6801, B′39011 |
| env | 117 | U455 | SF1703 [224] Z321 [219] 92RW020.5 [205] 92RW009.14 [217] TZ017 [210] D687 [105] UG275A [216] UG273 | 3 | B′39011, B′5101, Cw′0102 |
| env | 108 | U455 | SF1703 [423] 92RW020.5 [400] 92UG037.8 [410] UG275A [413] UG273A [417] C13271 [148]LBV2310 [153] | 3 | B′2705, B′39011, B′5801 |
| env | 50 | Z321 | D687 [298] K114 [164] L414 [152] P104 [145] PZ61 [143] C1211 [145] DJ264A [408] DJ263A [416] DJ258A [4 | 3 | B′2705, B′39011, B′5801 |
| env | 95 | SF2 | SF2813 [440] LAl [450] HXB2R [445] JB02 [169] NY5CG [437] NL43 [443] JRCSF [437] JRFL [436] ALA1 [431 | 3 | B7, B′39011, B′5801 |
| env | 114 | SF1703 | 92RW020.5 [283] 92UG037.8 [296] PZ61 [26] DJ264A [292] DJ263A [296] Cl31 [29] Cl451 [29] Cl3301 [29] L | 3 | A′0301, A′1101, B′5801 |
| env | 106 | US1 | US2 [558] CM237X [515] 91HT652.11 [556] 92UG005 [283] 3202A12 [564] 3202A21 [560] MANC [565] CA20 | 3 | B′39011, B′5101, B′5801 |
| env | 59 | 92UG021.16 | B H93TH067A [749] YU2 [753] JRFL [757] JRCSF [758] ALA1 [759] FB 93BR019.10 [760] NY5CG [760] AD | 3 | B14, B′39011, B′5801 |
| env | 62 | U455 | SF1703 [695] Z321 [690] 92RW020.5 [671] 92UG037.8 [683] D697 [572] UG275A [685] Vl191A [688] DJ263A | 3 | B′39011, B′5101, B′5801 |
| env | 98 | Z321 | A_GA1LBV23 [276] SF2 [547] SF2B13 [545] LAl [553] HXB2R [548] JB02 [275] NL43 [546] JRCSF [540] JRF | 4 | A′3101, A′3302, A′6801, B′39011 |
| env | 74 | U455 | SF1703 [553] 92RW020.5 [529] 92UG031.7 [547] 92UG037.8 [541] 92RW009.14 [543] P104 [277] Cl211 [275 | 4 | A′3101, A′3302, A′6801, B′39011 |
| env | 145 | SF1703 | 92UG031.7 [119] TZ017 [120] D687 [12] UG275A [120] UG273A [120] KENYA [120] CAR4054 [120] CAR4023 | 3 | A′0201, A′0301, B′39011 |
| env | 202 | U455 | SF1703 [116] Z321 [116] 92RW020.5 [114] 92UG031.7 [115] TZ017 [116] D687 [8] UG275A [116] UG273A [1 | 5 | B7, B35, B′39011, B′5101, B′5801 |
| env | 128 | U455 | 92UG031.7 [252] 92RW009.14 [251] D687 [139] K114 [1] UG08 [4] UG275A [250] Vl191A [253] DJ264A [248] | 5 | B7, B35, B′29011, B′5101, B′5801 |
| env | 50 | LAl | HXB2R [794] GP160EN [792] NL43 [792] JRCSF [786] JRFL [785] ALA1 [787] JH32 [805] BAL1 [794] YU2 [78 | 3 | A′0301, B′5801, Cw′0702 |
| env | 64 | SF2 | SF2B13 [658] LAl [666] HX62R [661] GP160EN [659] NY5CG [655] NL43 [659] JRCSF [653] JRFL [652] ALA1 | 3 | B40, B′4403, B′5801 |
| env | 92 | SF1703 | Z321 [687] 92RW020.5 [668] 92UG031.7 [686] 92UG037.8 [680] D687 [569] UG275A [682] UG273A [686] Vl1 | 3 | A′3101, A′3302, B′39011 |
| env | 54 | SF1703 | CARSAS [285] Z3 [277] I_GM4 [131] 93BR029.2 [281] F_H93BR028A [282] 92UG046.8 [283] 92UG038.1 [244 | 5 | B8, B35, B′5101, B′5801, Cw′0102 |
| env | 134 | TZ017 | CARSAS [87] CAR4054 [87] AD K124A2 [86] AD UG266A2 [87] CA ZAM184 [87] GX Vl525A2 [87] EA CA | 3 | A′0301, A′1101, A′6801 |
| env | 117 | U455 | UG275A [102] DJ264A [101] DJ263A [101] DJ25BA [101] CAR4054 [102] CAR423A [103] LAl [103] HX82R [1 | 4 | A′0201, A′0301, B′39011, B′5801 |
| env | 117 | U455 | SF1703 [562] Z321 [557] 92UG031.7 [556] 92UG037.8 [550] 92RW009.14 [552] C1211 [284] UG273A [556] D | 5 | A′0201, B7, B35, B′39011, B′5801 |
| env | 54 | LAl | HXB2R [444] JB02 [168] NY5CG [436] NL43 [442] JRCSF [436] JRFL [435] ALA1 [437] JH32 [456] BAL1 [442 | 3 | B7, B′39011, B′5801 |
| env | 94 | Z321 | 92UG037.8 [252] TZ017 [244] UG273A [256] CARSAS [257] A_MLY10A [133] LAl [257] HXB2R [252] GP1601 | 5 | B7, B35, B′39011, B′5101, B′5801 |
| env | 53 | CAR4054 | FB 93BR019.10 [475] BZ126A [466] RJl03 [347] 93BR020.17 [469] 93BR029.2 [466] AR16 [208] AR18 [200] | 3 | B40, B′4006, B′4006 |
| env | 129 | U455 | SF1703 [486] Z321 [481] 92RW020.5 [462] 92UG031.7 [480] 92RW009.14 [476] P104 [210] PZ61 [211] UG04 | 3 | B40, B′4006, B′4006 |
| env | 53 | 92RW009.14 | BF_RJl01.5 [162] CD_Dl2ACD [262] CAR4081 [265] U_BU91009A [262] RU570 [226] 93TH966.8 [264] E_92 | 3 | A′0301, A′3101, B′39011 |
| env | 55 | DJ264A | DJ263A [264] B H93TH067A [257] CB6 [141] CB7 [165] CB9 [141] US2 [265] 24612 [237] 26807 [253] 4995 [ | 3 | A′0301, A′3101, B′39011 |
| env | 66 | 92UG037.B | 92RW009.14 [410] DA_MAL [415] CA_ZAM184 [397] BF_RJl01.5 [306] FB_AR15 [133] HIV1UG3521 [406] R | 3 | B8, B′39011, Cw′0102 |
| env | 157 | U455 | SF1703 [36] Z321 [36] 92UG031.7 [35] 92UG037.8 [34] 92RW009.14 [34] TZ017 [36] KENYA [36] CARGAN [3 | 3 | A′0301, A′1101, A′6801 |

For example, the env peptide KLTPLCVTLN, conserved in 145 different strains on the LANL HIV sequence database, was selected from SF1703 (a clade B strain) and was conserved in SF2, SF2B13, 92UG031.7, TZ017, D687, UG275A, UG273A, CAR4054, CAR4023, CAR423A, A_MLY10A, NY5CG, JRCSF, JRFL, JH32, BAL1,YU2, BRVA, and more, representing several different clades. The HLA class I alleles for which the string is predicted to be a good (greater than 20%) ligand were A2, A0301, and B39.

Prior to selecting peptides for synthesis, we have analyzed the peptides for (1) representation of clade A, C, D and E strains, and (2) adequate representation of potential binding to HLA alleles that are prevalent in countries where clades A, C, D, and E are transmitted. Results from assays performed in the lab to date have shown that a very high proportion of the peptides we selected for our studies bound to T2 cells expressing the appropriate MHC in vitro.

TABLE 6
Aˆ 0101 PEPTIDE SEQUENCES

| | con- | | | | | |
|---|---|---|---|---|---|---|
| | ser- | | | | | SEQ |
| pro- | va- | | ref. | ref. | | ID. |
| tein | tion | sequence | strain | start | Aˆ 0101 | NO: |
| env | 107 | SFEPIPIHYC | U455 | 207 | 30.25% | 30 |
| env | 55 | ELDKWASLWN | US1 | 665 | 2.91% | 31 |
| env | 114 | CTRPNNNTRK | SF1703 | 302 | 1.31% | 332 |
| env | 61 | GVAPTKAKRR | Z321 | 495 | 0.89% | 33 |
| env | 126 | SFNCGGEFFY | U455 | 373 | 0.83% | 34 |
| env | 102 | ITLPCRIKQI | 92UG037.8 | 406 | 0.73% | 35 |
| env | 93 | SSNITGLLLT | AD_K124A2 | 448 | 0.70% | 36 |
| gag | 57 | RLRPGGKKKY | BNG | 20 | 11.73% | 37 |
| gag | 51 | AISPRTLNAW | BZ126B | 144 | 2.23% | 38 |
| gag | 32 | AWEKIRLRPG | BZ126B | 15 | 2.16% | 39 |
| gag | 53 | FRDYVDRFYK | TN243 | 293 | 2.03% | 40 |
| pol | 40 | LKEPVHGVYY | IBNG | 465 | 29.32% | 41 |
| pol | 44 | ETVPVKLKPG | IBNG | 161 | 12.68% | 42 |
| pol | 39 | ETPGIRYQYN | IBNG | 293 | 9.40% | 43 |
| pol | 46 | QKEPPFLWMG | U455 | 376 | 8.33% | 44 |
| pol | 39 | NNETPGIRYQ | IBNG | 291 | 3.29% | 45 |
| pol | 46 | TPDKKHQKEP | U455 | 370 | 3.19% | 46 |
| pol | 38 | IPHPAGLKKK | IBNG | 249 | 2.61% | 47 |
| pol | 43 | LVDFRELNKR | U455 | 228 | 2.23% | 48 |
| rev | 13 | SAEPVPLQLP | SF2 | 67 | 22.60% | 49 |
| tat | 7 | RGDPTGPKE$ | TH475A | 78 | 30.49% | 50 |
| vif | 17 | LADQLIHLYY | IBNG | 102 | 43.60% | 51 |
| vif | 10 | QVDPGLADQL | SF2 | 97 | 8.75% | 52 |
| vpr | 7 | LHSLGQHIYE | D31 | 39 | 0.60% | 53 |
| vpu | 35 | RAEDSGNESE | CM240X | 49 | 1.38% | 54 |

TABLE 7
Aˆ 0201 PEPTIDE SEQUENCES

| | | | | | | SEQ |
|---|---|---|---|---|---|---|
| pro- | conser- | | ref. | ref. | | ID. |
| tein | vation | sequence | strain | start | Aˆ 0201 | NO: |
| env | 91 | NLWVTVYYGV | Z321 | 32 | 82.51% | 55 |
| env | 110 | GIKQLQARVL | U455 | 565 | 72.16% | 56 |
| env | 91 | QLQARVLAVE | U455 | 568 | 63.81% | 57 |
| env | 145 | KLTPLCVTLN | SF1703 | 120 | 50.93% | 58 |
| env | 67 | NMWQEVGKAM | CA16 | 147 | 49.55% | 59 |
| env | 117 | QMHEDIISLW | U455 | 101 | 47.82% | 60 |
| env | 154 | DMRDNWRSEL | CA20 | 193 | 44.72% | 61 |
| gag | 31 | SLYNTVATLY | UG268 | 77 | 76.09% | 62 |
| gag | 25 | ELRSLYNTVA | U455 | 74 | 69.48% | 63 |
| gag | 88 | EMMTACQGVG | U455 | 341 | 63.81% | 64 |
| gag | 58 | DLNTMLNTVG | BZ126B | 181 | 63.81% | 65 |
| pol | 30 | LLWKGEGAVV | U455 | 955 | 99.50% | 66 |
| pol | 40 | ILKEPVHGVY | IBNG | 464 | 96.43% | 67 |
| pol | 27 | KLLWKGEGAV | U455 | 954 | 88.23% | 68 |
| pol | 28 | HLKTAVQMAV | U455 | 885 | 80.90% | 69 |
| pol | 39 | GLKKKKSVTV | U455 | 253 | 74.16% | 70 |
| pol | 48 | ELHPDKWTVQ | U455 | 387 | 70.39% | 71 |
| pol | 31 | KIEELRQHLL | SF2 | 356 | 69.18% | 72 |
| pol | 33 | KLLRGTKALT | SF2 | 436 | 61.17% | 73 |
| rev | 8 | QILVESPTVL | LAI | 101 | 67.94% | 74 |
| tat | 7 | FLNKGLGISY | UG275A | 38 | 10.68% | 75 |
| vif | 10 | DLADQLIHLY | IBNG | 101 | 54.04% | 76 |
| vif | 12 | HIPLGDARLV | IBNG | 56 | 46.44% | 77 |
| vpr | 9 | LLEELKNEAV | LAI | 22 | 87.89% | 78 |
| vpu | 7 | ILAIVVWTIV | U455 | 17 | 89.70% | 79 |

TABLE 8
Aˆ 0301 PEPTIDE SEQUENCES

| | | | | | | SEQ |
|---|---|---|---|---|---|---|
| pro- | conser- | | ref. | ref. | | ID |
| tein | vation | sequence | strain | start | | NO: |
| env | 129 | HSFNCGGEFF | U455 | 372 | 60.47% | 80 |
| env | 138 | TLFCASDAKA | U455 | 49 | 58.33% | 81 |
| env | 86 | HSFNCRGEFF | D687 | 259 | 55.44% | 82 |
| env | 174 | SLWDQSLKPC | U455 | 108 | 49.09% | 83 |
| env | 157 | TVYYGVPVWK | U455 | 35 | 48.61% | 84 |
| env | 93 | VSFEPIPIHY | U455 | 206 | 48.61% | 85 |
| env | 114 | CTRPNNNTRK | SF1703 | 302 | 43.25% | 86 |
| gag | 31 | SLYNTVATLY | UG268 | 77 | 49.34% | 87 |
| gag | 31 | LARNCRAPRK | BZ126B | 399 | 32.34% | 88 |
| gag | 57 | RLRPGGKKKY | BNG | 20 | 32.12% | 89 |
| gag | 73 | ILDIRQGPKE | U455 | 278 | 29.11% | 90 |
| pol | 43 | LVDFRELNKR | U455 | 228 | 52.52% | 91 |
| pol | 27 | QLDCTHLEGK | U455 | 776 | 50.32% | 92 |
| pol | 27 | AVFIHNEKRK | U455 | 893 | 43.98% | 93 |
| pol | 38 | QIIEQLIKKE | SF2 | 675 | 43.01% | 94 |
| pol | 40 | GIPHPAGLKK | IBNG | 248 | 41.81% | 95 |
| pol | 39 | KVYLAWVPAH | SF2 | 685 | 36.86% | 96 |
| pol | 35 | AIFQSSMTKI | SF2 | 313 | 34.57% | 97 |
| pol | 46 | KLVDFRELNK | U455 | 227 | 33.45% | 98 |
| rev | 6 | KILYQSNPYP | UG273A | 20 | 23.70% | 99 |
| tat | 7 | TACNNCYCKK | SF2 | 20 | 62.35% | 100 |
| vif | 6 | ALTALITPKK | MN | 149 | 37.32% | 101 |
| vif | 31 | KLTEDRWNKP | U455 | 168 | 35.02% | 102 |
| vpr | 27 | WTLELLEELK | IBNG | 18 | 22.76% | 103 |
| vpu | 9 | RLIDRIRERA | SC | 42 | 37.32% | 104 |

TABLE 9
Aˆ 1101 PEPTIDE SEQUENCES

| | | | | | | SEQ |
|---|---|---|---|---|---|---|
| pro- | conser- | | ref. | ref | | ID |
| tein | vation | sequence | strain | start | | NO: |
| env | 101 | TVQCTHGIKP | U455 | 242 | 52.33% | 105 |
| env | 51 | FAILKCNDKK | BF_RJI01.5 | 121 | 45.11% | 106 |
| env | 134 | NVTENFNMWK | TZ017 | 87 | 38.39% | 107 |
| env | 62 | TITLPCRIKQ | 92UG037.8 | 405 | 38.05% | 108 |
| env | 157 | TVYYGVPVWK | U455 | 35 | 33.47% | 109 |
| env | 114 | CTRPNNNTRK | SF1703 | 302 | 33.05% | 110 |
| env | 135 | VTENFNMWKN | TZ017 | 88 | 32.62% | 111 |
| gag | 57 | IRLRPGGKKK | BNG | 19 | 57.42% | 112 |
| gag | 64 | KIRLRPGGKK | BZ126B | 18 | 47.32% | 113 |
| gag | 91 | LVQNANPDCK | U455 | 318 | 33.37% | 114 |
| gag | 43 | ARNCRAPRKK | BZ126B | 400 | 25.16% | 115 |
| pol | 38 | FTTPDKKHQK | IBNG | 369 | 64.26% | 116 |
| pol | 40 | GIPHPAGLKK | IBNG | 248 | 63.28% | 117 |
| pol | 43 | TTPDKKHQKE | IBNG | 370 | 62.39% | 118 |
| pol | 38 | IPHPAGLKKK | IBNG | 249 | 58.91% | 119 |
| pol | 27 | AVFIHNFKRK | U455 | 893 | 57.99% | 120 |
| pol | 40 | NTPVFAIKKK | U455 | 211 | 57.88% | 121 |
| pol | 45 | PGMDGPKVKQ | IBNG | 169 | 57.65% | 122 |
| pol | 27 | QVRDQAEHLK | IBNG | 879 | 55.58% | 123 |
| rev | 9 | PTVLESGTKE | LAI | 107 | 31.68% | 124 |
| tat | 7 | TACNNCYCKK | SF2 | 20 | 70.97% | 125 |
| vif | 6 | IKPPLPSVKK | MN | 159 | 51.98% | 126 |
| vif | 6 | ALTALITPKK | MN | 149 | 44.77% | 127 |
| vpr | 27 | WTLELLEELK | IBNG | 18 | 21.41% | 128 |
| vpu | 8 | WTIVFIEYRK | CDC42 | 23 | 31.58% | 129 |

TABLE 10
Aˆ 2401 PEPTIDE SEQUENCES

| | | | | | | SEQ |
|---|---|---|---|---|---|---|
| pro- | conser- | | ref. | ref. | | ID |
| tein | vation | sequence | strain | start | Aˆ 2401 | NO: |
| env | 67 | RYLKDQQLLG | SF1703 | 590 | 58.82% | 130 |
| env | 58 | SYHRLRDLLL | DA_MAL | 770 | 0.18% | 131 |
| pol | 38 | IYQEPFKNLK | U455 | 495 | 15.49% | 132 |
| pol | 27 | VYYDPSKDLI | LAI | 484 | 0.01% | 133 |
| vif | 17 | YYFDCFSESA | JRCSF | 110 | 0.02% | 134 |
| vpr | 18 | PYNEWTLELL | SF2 | 14 | 0.01% | 135 |

TABLE 11
Aˆ 3101 PEPTIDE SEQUENCES

| | | | | | Aˆ 3101 | SEQ |
|---|---|---|---|---|---|---|
| pro- | conser- | | ref. | ref. | (10- | ID |
| tein | vation | sequence | strain | start | mers) | NO: |
| env | 92 | MIVGGLIGLR | SF1703 | 692 | 71.89% | 136 |
| env | 53 | SLAEEEIIIR | 92RW009.14 | 263 | 71.89% | 137 |
| env | 98 | IVQQQNNLLR | Z321 | 548 | 39.79% | 138 |
| env | 74 | IVQQQSNLLR | U455 | 541 | 39.79% | 139 |
| env | 55 | SLAEEEVVIR | DJ264A | 260 | 39.79% | 140 |
| env | 101 | STVQCTHGIR | SF1703 | 249 | 13.63% | 141 |
| env | 83 | LQARVLAVER | U455 | 569 | 13.63% | 142 |
| gag | 42 | LVWASRELER | BNG | 34 | 85.94% | 143 |
| gag | 37 | IVWASRELER | K98 | 34 | 85.94% | 144 |
| gag | 89 | IILGLNKIVR | U455 | 262 | 71.89% | 145 |
| gag | 44 | QMVHQAISPR | BZ126B | 139 | 71.89% | 146 |
| pol | 27 | KIQNFRVYYR | U455 | 933 | 99.88% | 147 |
| pol | 43 | LVDFRELNKR | U455 | 228 | 39.79% | 148 |
| pol | 46 | KLVDFRELNK | U455 | 227 | 18.66% | 149 |
| pol | 40 | SMTKILEPFR | U455 | 317 | 13.63% | 150 |
| pol | 29 | SINNETPGIR | SF2 | 289 | 13.63% | 151 |
| pol | 26 | GIGGYSAGER | U455 | 904 | 13.63% | 152 |
| pol | 39 | TFYVDGAANR | U455 | 593 | 11.15% | 153 |
| pol | 30 | SQIIEQLIKK | SF2 | 674 | 8.24% | 154 |
| rev | 34 | GTRQARRNRR | SF2 | 33 | 2.65% | 155 |
| tat | 10 | KTACTNCYCK | HXB2R | 19 | 7.36% | 156 |
| vif | 6 | AILGHIVSPR | JRCSF | 123 | 71.89% | 157 |
| vif | 33 | QVMIVWQVDR | U455 | 6 | 59.46% | 158 |
| vpr | 27 | LQQLLFIHFR | U455 | 64 | 39.79% | 159 |
| vpu | 21 | KILRQRKIDR | CM240X | 32 | 97.23% | 160 |

TABLE 12
Aˆ 3302 PEPTIDE SEQUENCES

| | | | | | | SEQ |
|---|---|---|---|---|---|---|
| pro- | conser- | | ref. | ref. | A*3302 | ID |
| tein | vation | sequence | strain | start | (10-mers) | NO: |
| env | 51 | EITTHSFNCR | UG23 | 93 | 76.02% | 161 |
| env | 98 | IVQQQNNLLR | Z321 | 548 | 23.98% | 162 |
| env | 92 | MIVGGLIGLR | SF1703 | 692 | 23.98% | 163 |
| env | 91 | ASITLTVQAR | U455 | 526 | 23.98% | 164 |
| env | 82 | AIAVAEGTDR | SF2B13 | 816 | 23.98% | 165 |
| env | 74 | IVQQQSNLLR | U455 | 541 | 23.98% | 166 |
| env | 69 | AVLSIVNRVR | SF2 | 699 | 23.98% | 167 |
| gag | 89 | IILGLNKIVR | U455 | 262 | 23.98% | 168 |
| gag | 62 | GVGGPGHKAR | U455 | 348 | 23.98% | 169 |
| gag | 52 | YVDRFYKTLR | ELI | 240 | 23.98% | 170 |
| gag | 48 | YSPVSILDIR | ZAM19 | 157 | 23.98% | 171 |
| pol | 27 | ELKKIIGQVR | U455 | 871 | 52.05% | 172 |
| pol | 43 | LVDFRELNKR | U455 | 228 | 23.98% | 173 |
| pol | 42 | GSDLEIGQHR | U455 | 344 | 23.98% | 174 |
| pol | 40 | SMTKILEPFR | U455 | 317 | 23.98% | 175 |
| pol | 29 | SINNETPGIR | SF2 | 289 | 23.98% | 176 |
| pol | 26 | GIGGYSAGER | U455 | 904 | 23.98% | 177 |
| pol | 45 | EAELELAENR | U455 | 452 | 8.65% | 178 |
| pol | 27 | KIQNFRVYYR | U455 | 933 | 1.22% | 179 |
| rev | 32 | EGTRQARRNR | SF2 | 32 | 8.65% | 180 |
| tat | 47 | GISYGRKKRR | DJ263A | 44 | 23.98% | 181 |
| vif | 12 | EVHIPLGDAR | IBNG | 54 | 76.02% | 182 |
| vif | 33 | QVMIVWQVDR | U455 | 6 | 23.98% | 183 |
| vpr | 7 | HSRIGITRQR | JRCSF | 78 | 23.98% | 184 |
| vpu | 6 | DSGNESEGDR | ELI | 52 | 76.02% | 185 |

TABLE 13
Aˆ 6801 PEPTIDE SEQUENCES

| | | | | | | SEQ |
|---|---|---|---|---|---|---|
| pro- | conser- | | ref. | ref | A*6801 | ID |
| tein | vation | sequence | strain | start | (10-mers) | NO: |
| env | 61 | GVAPTKAKRR | Z321 | 495 | 65.96% | 186 |
| env | 69 | AVLSIVNRVR | SF2 | 699 | 54.21% | 187 |
| env | 98 | IVQQQNNLLR | Z321 | 548 | 34.15% | 188 |
| env | 74 | IVQQQSNLLR | U455 | 541 | 34.15% | 189 |
| env | 157 | TVYYGVPVWK | U455 | 35 | 21.52% | 190 |
| env | 134 | NVTENFNMWK | TZ017 | 87 | 21.52% | 191 |
| env | 101 | STVQCTHGIR | SF1703 | 249 | 17.62% | 192 |
| gag | 62 | GVGGPGHKAR | U455 | 348 | 54.21% | 193 |
| gag | 26 | GVGGPSHKAR | V1310 | 351 | 54.21% | 194 |
| gag | 42 | LVWASRELER | BNG | 34 | 45.90% | 195 |
| gag | 37 | IVWASRELER | K98 | 34 | 45.90% | 196 |
| pol | 27 | AVFIHNEKRK | U455 | 893 | 39.20% | 197 |
| pol | 43 | LVDFRELNKR | U455 | 228 | 34.15% | 198 |
| pol | 32 | LVEICTEMEK | SF2 | 189 | 31.46% | 199 |
| pol | 27 | QVRDQAEHLK | IBNG | 879 | 31.46% | 200 |
| pol | 42 | LVKLWYQLEK | U455 | 576 | 21.52% | 201 |
| pol | 38 | FTTPDKKHQK | IBNG | 369 | 6.44% | 202 |
| pol | 35 | DSWTVNDIQK | U455 | 404 | 5.56% | 203 |
| pol | 40 | NTPVFAIKKK | U455 | 211 | 3.41% | 204 |
| rev | 34 | GTRQARRNRR | SF2 | 33 | 7.44% | 205 |
| tat | 10 | KTACTNCYCK | HXB2R | 19 | 9.51% | 206 |
| vif | 12 | EVHIPLGDAR | IBNG | 54 | 65.96% | 207 |
| vif | 33 | QVMIVWQVDR | U455 | 6 | 54.21% | 208 |
| vpr | 27 | WTLELLEELK | IBNG | 18 | 15.76% | 209 |
| vpu | 6 | DSGNESEGDR | ELI | 52 | 24.23% | 210 |

TABLE 14
B7 PEPTIDE SEQUENCES

| | | | | | | SEQ |
|---|---|---|---|---|---|---|
| pro- | conser- | | ref. | ref. | | ID |
| tein | vation | sequence | strain | start | B7 | NO: |
| env | 128 | KPVVSTQLLL | U455 | 250 | 67.23% | 211 |
| env | 94 | RPVVSTQLLL | Z321 | 253 | 62.56% | 212 |
| env | 202 | KPCVKLTPLC | U455 | 115 | 43.65% | 213 |
| env | 54 | RCSSNITGLL | LAI | 449 | 32.95% | 214 |
| env | 84 | APTKAKRRVV | Z321 | 497 | 30.13% | 215 |
| env | 117 | RAIEAQQHLL | U455 | 550 | 28.51% | 216 |
| env | 72 | GPCKNVSTVQ | SF1703 | 243 | 25.30% | 217 |
| gag | 58 | TPQDLNTMLN | UG268 | 175 | 50.10% | 218 |
| gag | 30 | TPQDLNMMLN | AD_K124 | 180 | 49.09% | 219 |
| gag | 60 | GPGHKARVLA | U455 | 351 | 45.50% | 220 |
| gag | 74 | APRKKGCWKC | U455 | 401 | 38.60% | 221 |
| pol | 32 | QPDKSESELV | SF2 | 664 | 55.70% | 222 |
| pol | 43 | GPKVKQWPLT | U455 | 172 | 43.22% | 223 |
| pol | 34 | SPAIFQSSMT | SF2 | 311 | 21.23% | 224 |
| pol | 44 | SPIETVPVKL | U455 | 157 | 18.90% | 225 |
| pol | 31 | KIEELRQHLL | SF2 | 356 | 17.10% | 226 |
| pol | 27 | QVRDQAEHLK | IBNG | 879 | 16.74% | 227 |
| pol | 28 | LVSQIIEQLI | SF2 | 672 | 11.11% | 228 |
| pol | 29 | IPAETGQETA | U455 | 803 | 11.04% | 229 |
| rev | 23 | LPPLERLTLD | SF2 | 75 | 68.27% | 230 |
| tat | 8 | GPKE$KKKVE | TH475A | 83 | 14.25% | 231 |
| vif | 7 | KPPLPSVTKL | LAI | 160 | 43.22% | 232 |
| vif | 10 | KPPLPSVKKL | U455 | 160 | 38.19% | 233 |
| vpr | 11 | FPRIWLHSLG | JRCSF | 34 | 65.66% | 234 |
| vpu | 6 | LVILAIVALV | TZ012 | 4 | 8.00% | 235 |

TABLE 15
B8 PEPTIDE SEQUENCES

| | | | | | | SEQ |
|---|---|---|---|---|---|---|
| pro- | conser- | | | ref. | | ID |
| tein | vation | sequence | ref. strain | start | B8 | NO: |
| env | 54 | NAKTIIVQLN | SF1703 | 286 | 36.95% | 236 |
| env | 56 | PTKAKRRVVQ | SF2 | 496 | 36.67% | 237 |
| env | 119 | LYKYKVVKIE | U455 | 476 | 32.46% | 238 |
| env | 66 | TLPCRIKQII | 92UG037.8 | 407 | 24.36% | 239 |
| env | 105 | VPVWKEATTT | SF2 | 41 | 23.42% | 240 |
| env | 131 | VWGIKQLQAR | U455 | 563 | 21.82% | 241 |
| env | 64 | DAKAYDTEVH | 92RW020.5 | 54 | 20.93% | 242 |
| gag | 43 | FNCGKEGHLA | U455 | 387 | 26.43% | 243 |
| gag | 39 | NAWVKVVEEK | BZ126B | 151 | 20.49% | 244 |
| gag | 47 | DCKTILKALG | SF2 | 331 | 19.96% | 245 |
| gag | 49 | NAWVKVIEEK | BNG | 150 | 19.32% | 246 |
| pol | 39 | GLKKKKSVTV | U455 | 253 | 73.44% | 247 |
| pol | 43 | GPKVKQWPLT | U455 | 172 | 72.05% | 248 |
| pol | 46 | AIKKKDSTKW | U455 | 216 | 51.14% | 249 |
| pol | 46 | FAIKKKDSTK | U455 | 215 | 49.32% | 250 |
| pol | 36 | QHRTKIEELR | SF2 | 352 | 43.87% | 251 |
| pol | 27 | ELKKIIGQVR | U455 | 871 | 35.67% | 252 |
| pol | 38 | AGLKKKKSVT | U455 | 252 | 25.94% | 253 |
| pol | 26 | GIKVKQLCKL | U455 | 427 | 25.33% | 254 |
| rev | 7 | IIKILYQSNP | UG273A | 18 | 7.75% | 255 |
| tat | 16 | ESKKKVERET | SF2 | 86 | 65.88% | 256 |
| vif | 9 | TPKKIKPPLP | LAI | 155 | 22.95% | 257 |
| vif | 27 | AGHNKVGSLQ | U455 | 137 | 22.95% | 258 |
| vpr | 22 | EAIIRILQQL | U455 | 58 | 19.22% | 259 |
| vpu | 7 | WLIDRIRERA | TZ023 | 41 | 6.13% | 260 |

TABLE 16
B14 PEPTIDE SEQUENCES

| | | | | | | SEQ |
|---|---|---|---|---|---|---|
| pro- | conser- | | | ref. | | ID |
| tein | vation | sequence | ref. strain | start | B14 | NO: |
| env | 68 | ERYLKDQQLL | US2 | 582 | 97.12% | 261 |
| env | 59 | FSYHRLRDLL | 92UG021.16 | 749 | 20.43% | 262 |
| env | 106 | EAQQHLLQLT | US1 | 562 | 9.22% | 263 |
| env | 178 | MRDNWRSELY | SF1703 | 480 | 0.35% | 264 |
| env | 50 | CRIKQIVNMW | Z321 | 418 | 0.28% | 265 |
| env | 56 | PTKAKRRVVQ | SF2 | 496 | 0.16% | 266 |
| env | 66 | TLPCRIKQII | 92UG037.8 | 407 | 0.13% | 267 |
| gag | 37 | DRFFKTLRAE | U455 | 294 | 44.20% | 268 |
| gag | 52 | DRFYKTLRAE | TN243 | 298 | 36.29% | 269 |
| gag | 26 | ERFAVNPGLL | SF2 | 42 | 5.50% | 270 |
| gag | 31 | SLYNTVATLY | UG268 | 77 | 0.25% | 271 |
| pol | 32 | GAANRETKLG | U455 | 598 | 0.40% | 272 |
| pol | 31 | NRETKLGKAG | U455 | 601 | 0.08% | 273 |
| pol | 45 | KLVGKLNWAS | U455 | 413 | 0.03% | 274 |
| pol | 30 | EPFRKQNPDI | SF2 | 324 | 0.01% | 275 |
| pol | 33 | LTEEKIKALV | SF2 | 181 | 0.01% | 276 |
| pol | 44 | WTVNDIQKLV | U455 | 406 | 0.01% | 277 |
| rev | 35 | TRQARRNRRR | SF2 | 34 | 4.66% | 278 |
| tat | 35 | GRKKRRQRRR | SF2 | 48 | 2.30% | 279 |
| vif | 27 | DRWNKPQKTK | SF2 | 172 | 53.54% | 280 |
| vif | 22 | ERDWHLGQGV | IFA86 | 76 | 6.68% | 281 |
| vpr | 6 | QREPHNEWTL | LAI | 11 | 1.91% | 282 |
| vpu | 19 | LRQRKIDRLI | LAI | 33 | 4.71% | 283 |

TABLE 17
Bˆ 1501 (10-mers) PEPTIDE SEQUENCES

| | | | | | | SEQ |
|---|---|---|---|---|---|---|
| pro- | conser- | | | ref. | Bˆ 1501 | ID |
| tein | vation | sequence | ref. strain | start | (10-mers) | NO: |
| env | 93 | DLRSLCLFSY | DJ259A | 735 | 66.56% | 284 |
| env | 101 | QQHLLQLTVW | SF2 | 561 | 0.47% | 285 |
| gag | 57 | RLRPGGKKKY | BNG | 20 | 36.98% | 286 |
| gag | 31 | SLYNTVATLY | UG268 | 77 | 2.43% | 287 |
| gag | 71 | DIRQGPKEPF | U455 | 280 | 0.38% | 288 |
| gag | 83 | RQANFLGKIW | U455 | 423 | 0.13% | 289 |
| pol | 40 | ILKEPVHGVY | IBNG | 464 | 53.38% | 290 |
| pol | 33 | GQGQWTYQIY | SF2 | 488 | 42.73% | 291 |
| pol | 28 | VQMAVFIHNF | U455 | 890 | 42.73% | 292 |
| pol | 44 | IQKLVGKLNW | U455 | 411 | 4.02% | 293 |
| pol | 38 | EQLIKKEKVY | SF2 | 678 | 1.83% | 294 |
| pol | 47 | YQYNVLPQGW | U455 | 298 | 0.13% | 295 |
| pol | 46 | HQKEPPFLWM | U455 | 375 | 0.01% | 296 |
| rev | 11 | LLKTVRLIKF | MN | 12 | 75.68% | 297 |
| tat | 7 | FLNKGLGISY | UG275A | 38 | 17.27% | 298 |
| vif | 10 | DLADQLIHLY | IBNG | 101 | 1.83% | 299 |
| vif | 23 | HLGQGVSIEW | IFA86 | 80 | 0.30% | 300 |
| vpr | 23 | ILQQLLFIHF | U455 | 63 | 28.91% | 301 |

TABLE 18
Bˆ 2705 PEPTIDE SEQUENCES

| pro- | conser- | | | ref. | | SEQ ID |
|---|---|---|---|---|---|---|
| tein | vation | sequence | ref. strain | start | Bˆ 2705 | NO: |
| env | 108 | CRIKQIINMW | U455 | 411 | 94.41% | 302 |
| env | 50 | CRIKQIVNMW | Z321 | 418 | 85.77% | 303 |
| env | 82 | RRVVQREKRA | SF1703 | 508 | 16.62% | 304 |
| env | 88 | KRRVVQREKR | SF1703 | 507 | 13.63% | 305 |
| env | 103 | RRVVEREKRA | U455 | 496 | 12.89% | 306 |
| env | 51 | IRSENLTNNA | CI3301 | 5 | 12.89% | 307 |
| env | 90 | KRRVVEREKR | U455 | 495 | 7.04% | 308 |
| gag | 81 | KRWIILGLNK | BZ126B | 261 | 25.12% | 309 |
| gag | 71 | IRQGPKEPFR | U455 | 281 | 14.39% | 310 |
| gag | 57 | IRLRPGGKKK | BNG | 19 | 12.19% | 311 |
| gag | 43 | ARNCRAPRKK | BZ126B | 400 | 8.94% | 312 |
| pol | 26 | KRKGGIGGYS | U455 | 900 | 33.92% | 313 |
| pol | 38 | KRTQDFWEVQ | U455 | 236 | 5.76% | 314 |
| pol | 30 | HRTKIEELRQ | SF2 | 353 | 0.61% | 315 |
| pol | 27 | KQNPDIVIYQ | SF2 | 328 | 0.37% | 316 |
| pol | 26 | VRDQAEHLKT | IBNG | 880 | 0.30% | 317 |
| pol | 40 | IRYQYNVLPQ | IBNG | 297 | 0.13% | 318 |
| pol | 29 | KALTEVIPLT | SF2 | 442 | 0.11% | 319 |
| pol | 37 | WGFTTPDKKH | IBNG | 367 | 0.09% | 320 |
| rev | 13 | GRSAEPVPLQ | SF2 | 65 | 47.75% | 321 |
| tat | 9 | RRAPQDSQTH | SF2 | 56 | 13.07% | 322 |
| vif | 32 | NRWQVMIVWQ | U455 | 3 | 10.24% | 323 |
| vif | 11 | ARLVITTYWG | LAI | 62 | 8.14% | 324 |
| vpr | 6 | SRIGIIQQRR | SF2 | 79 | 97.28% | 325 |
| vpu | 19 | LRQRKIDRLI | LAI | 33 | 0.63% | 326 |

TABLE 19
B35 PEPTIDE SEQUENCES

| pro- | conser- | | | ref. | | SEQ ID |
|---|---|---|---|---|---|---|
| tein | vation | sequence | ref. strain | start | B35 | NO: |
| env | 202 | KPCVKLTPLC | U455 | 115 | 94.43% | 327 |
| env | 128 | KPVVSTQLLL | U455 | 250 | 94.43% | 328 |
| env | 94 | RPVVSTQLLL | Z321 | 253 | 94.43% | 329 |
| env | 100 | CPKVSFEPIP | U455 | 203 | 83.30% | 330 |
| env | 117 | RAIEAQQHLL | U455 | 550 | 53.09% | 331 |
| env | 54 | NAKTIIVQLN | SF1703 | 286 | 39.25% | 332 |
| env | 85 | LPCRIKQIIN | SF1703 | 421 | 34.07% | 333 |
| gag | 92 | GPKEPFRDYV | U455 | 284 | 99.99% | 334 |
| gag | 32 | GPAATLEEMM | LBV2310 | 335 | 94.57% | 335 |
| gag | 31 | GPGATLEEMM | U455 | 334 | 94.57% | 336 |
| gag | 58 | TPQDLNTMLN | UG268 | 175 | 94.43% | 337 |
| pol | 43 | GPKVKQWPLT | U455 | 172 | 98.24% | 338 |
| pol | 46 | VPVKLKPGMD | IBNG | 163 | 94.57% | 339 |
| pol | 46 | EPPFLWMGYE | U455 | 378 | 94.57% | 340 |
| pol | 44 | TPPLVKLWYQ | U455 | 573 | 94.57% | 341 |
| pol | 34 | SPAIFQSSMT | SF2 | 311 | 94.57% | 342 |
| pol | 28 | EPIVGAETFY | SF2 | 587 | 76.68% | 343 |
| pol | 27 | NPDIVIYQYM | SF2 | 330 | 54.09% | 344 |
| pol | 45 | KPGMDGPKVK | IBNG | 168 | 53.59% | 345 |
| rev | 23 | LPPLERLTLD | SF2 | 75 | 89.28% | 346 |
| tat | 14 | GPKESKKKVE | SF170 | 83 | 82.99% | 347 |
| vif | 9 | TPKKIKPPLP | LAI | 155 | 98.24% | 348 |
| vif | 12 | KSLVKHHMYI | SF2 | 22 | 76.68% | 349 |
| vpr | 11 | FPRIWLHSLG | JRCSF | 34 | 98.24% | 350 |
| vpu | 6 | QPLVILAIVA | TZ023 | 2 | 9.91% | 351 |

TABLE 20
B38 PEPTIDE SEQUENCES

| | | | | | | SEQ |
|---|---|---|---|---|---|---|
| pro- | conser- | | | ref. | | ID |
| tein | vation | sequence | ref. strain | start | B38 | NO: |
| env | 121 | IHYCAPAGFA | U455 | 213 | 55.70% | 352 |
| env | 115 | MHEDIISLWD | U455 | 102 | 46.23% | 353 |
| env | 59 | YHRLRDLLLI | LAI | 773 | 23.31% | 354 |
| env | 101 | QHLLQLTVWG | SF2 | 562 | 9.57% | 355 |
| env | 119 | THGIKPVVST | U455 | 246 | 9.29% | 356 |
| env | 97 | THGIRPVVST | Z321 | 249 | 9.19% | 357 |
| env | 129 | VHNVWATHAC | U455 | 63 | 9.01% | 358 |
| gag | 95 | GHQAAMQMLK | U455 | 189 | 57.48% | 359 |
| gag | 35 | SHKGRPGNFL | SM145 | 436 | 38.92% | 360 |
| gag | 28 | LHPVHAGPIA | BZ167 | 216 | 23.66% | 361 |
| gag | 45 | VHQAISPRTL | SM145 | 140 | 12.44% | 362 |
| pol | 34 | AHTNDVKQLT | U455 | 514 | 50.97% | 363 |
| pol | 46 | KHQKEPPFLW | U455 | 374 | 47.58% | 364 |
| pol | 36 | QHRTKIEELR | SF2 | 352 | 25.26% | 365 |
| pol | 28 | EHLKTAVQMA | U455 | 884 | 19.21% | 366 |
| pol | 31 | KIEELRQHLL | SF2 | 356 | 14.26% | 367 |
| pol | 32 | QPDKSESELV | SF2 | 664 | 13.64% | 368 |
| pol | 35 | LTEEAELELA | U455 | 449 | 13.51% | 369 |
| pol | 33 | LTEEKIKALV | SF2 | 181 | 10.36% | 370 |
| rev | 13 | SAEPVPLQLP | SF2 | 67 | 13.03% | 371 |
| tat | 21 | KHPGSQPKTA | TH475A | 12 | 22.79% | 372 |
| vif | 18 | IHLYYFDCFS | LAI | 107 | 48.94% | 373 |
| vif | 8 | IHLHYFDCFS | U455 | 107 | 48.94% | 374 |
| vpr | 6 | PHNEWTLELL | LAI | 14 | 17.41% | 375 |
| vpu | 19 | ESEGDQEELS | SF2 | 56 | 10.36% | 376 |

TABLE 21
Bˆ 39011 PEPTIDE SEQUENCES

| | | | | | | SEQ |
|---|---|---|---|---|---|---|
| pro- | conser- | | ref. | ref. | | ID |
| tein | vation | sequence | strain | start | B*39011 | NO: |
| env | 115 | MHEDIISLWD | U455 | 102 | 58.82% | 377 |
| env | 178 | MRDNWRSELY | SF1703 | 480 | 56.02% | 378 |
| env | 108 | CRIKQIINMW | U455 | 411 | 49.57% | 379 |
| env | 93 | IRPVVSTQLL | Z321 | 252 | 49.57% | 380 |
| env | 50 | CRIKQIVNMW | Z321 | 418 | 49.57% | 381 |
| env | 68 | ERYLKDQQLL | US2 | 582 | 49.57% | 382 |
| env | 59 | YHRLRDLLLI | LAI | 773 | 48.00% | 383 |
| gag | 95 | GHQAAMQMLK | U455 | 189 | 80.51% | 384 |
| gag | 28 | LHPVHAGPIA | BZ167 | 216 | 60.35% | 385 |
| gag | 26 | ERFAVNPGLL | SF2 | 42 | 60.35% | 386 |
| gag | 38 | SRELERFALN | SM145 | 38 | 56.02% | 387 |
| pol | 34 | AHTNDVKQLT | U455 | 514 | 80.51% | 388 |
| pol | 46 | KHQKEPPFLW | U455 | 374 | 75.73% | 389 |
| pol | 28 | EHLKTAVQMA | U455 | 884 | 70.38% | 390 |
| pol | 36 | QHRTKIEELR | SF2 | 352 | 64.99% | 391 |
| pol | 33 | LTEEKIKALV | SF2 | 181 | 58.82% | 392 |
| pol | 27 | VYYDPSKDLI | LAI | 484 | 45.95% | 393 |
| pol | 44 | WTVNDIQKLV | U455 | 406 | 41.59% | 394 |
| pol | 43 | GGNEQVDKLV | U455 | 697 | 41.59% | 395 |
| rev | 13 | GRSAEPVPLQ | SF2 | 65 | 49.57% | 396 |
| tat | 6 | ERETETDPVH | BAL1 | 92 | 49.57% | 397 |
| vif | 23 | WHLGQGVSIE | IFA86 | 79 | 70.38% | 398 |
| vif | 9 | THPRISSEVH | MN | 47 | 60.35% | 399 |
| vpr | 27 | WTLELLEELK | IBNG | 18 | 52.41% | 400 |
| vpu | 19 | LRQRKIDRLI | LAI | 33 | 56.02% | 401 |

TABLE 22
B40 PEPTIDE SEQUENCES

| | | | | | | SEQ |
|---|---|---|---|---|---|---|
| pro- | conser- | | | ref. | | ID |
| tein | vation | sequence | ref. strain | start | B40 | NO: |
| env | 85 | QEVGKAMYAP | SF2 | 425 | 60.96% | 402 |
| env | 69 | VELLGRRGWE | LAI | 787 | 48.24% | 403 |
| env | 64 | LELDKWASLW | SF2 | 660 | 48.24% | 404 |
| env | 51 | GEFFYCNTSG | U455 | 378 | 44.21% | 405 |
| env | 100 | TEVHNVWATH | 92UG037.8 | 60 | 32.15% | 406 |
| env | 129 | SELYKYKVVK | U455 | 474 | 21.60% | 407 |
| env | 101 | KEATTTLFCA | SF2 | 45 | 21.60% | 408 |
| gag | 29 | IEVKDTKEAL | BZ126B | 92 | 60.96% | 409 |
| gag | 58 | EEAAEWDRLH | U455 | 203 | 48.24% | 410 |
| gag | 51 | GEIYKRWIIL | BZ126B | 257 | 44.21% | 411 |
| gag | 95 | REPRGSDIAG | U455 | 225 | 35.87% | 412 |
| pol | 43 | WEFVNTPPLV | U455 | 568 | 60.96% | 413 |
| pol | 44 | AETFYVDGAA | U455 | 591 | 48.24% | 414 |
| pol | 27 | TELQAIHLAL | SF2 | 632 | 48.24% | 415 |
| pol | 35 | LEVNIVTDSQ | SF2 | 646 | 32.15% | 416 |
| pol | 48 | YELHPDKWTV | U455 | 386 | 27.53% | 417 |
| pol | 38 | NDVKQLTEAV | SF2 | 518 | 24.83% | 418 |
| pol | 36 | TEEAELELAE | U455 | 450 | 24.83% | 419 |
| pol | 40 | GDAYFSVPLD | U455 | 266 | 24.68% | 420 |
| rev | 11 | EELLKTVRLI | MN | 10 | 48.24% | 421 |
| tat | 31 | LEPWKHPGSQ | U455 | 8 | 13.49% | 422 |
| vif | 15 | IEWRKKRYST | LAI | 87 | 21.60% | 423 |
| vif | 8 | IEWRKRRYST | HAN | 88 | 21.60% | 424 |
| vpr | 19 | YETYGDTWAG | SF2 | 47 | 35.87% | 425 |
| vpu | 17 | VEMGHHAPWD | LAI | 68 | 48.24% | 426 |

TABLE 23
Bˆ 40012 PEPTIDE SEQUENCE

| pro- | conser- | | ref. | ref. | | SEQ ID |
|---|---|---|---|---|---|---|
| tein | vation | sequence | strain | start | B*40012 | NO: |
| rev | 11 | EELLKTVRLI | MN | 10 | 71.53% | 427 |

TABLE 24
Bˆ 4006 (8mers) PEPTIDE SEQUENCES

| | con- | | | | | |
|---|---|---|---|---|---|---|
| | ser- | | | | | SEQ |
| pro- | va- | | | ref. | B*4006 | ID |
| tein | tion | sequence | ref. strain | start | (8-mers) | NO: |
| env | 53 | SELYKYKVVE | CAR4054 | 476 | 65.30% | 428 |
| env | 129 | SELYKYKVVK | U455 | 474 | 65.30% | 429 |
| env | 100 | TEVHNVWATH | 92UG037.8 | 60 | 23.25% | 430 |
| env | 51 | GEFFYCNTSG | U455 | 378 | 8.34% | 431 |
| env | 106 | IEAQQHLLQL | SF2 | 558 | 8.00% | 432 |
| env | 73 | REKRAVGIGA | SF1703 | 513 | 5.40% | 433 |
| env | 96 | VEQMHEDIIS | UG275A | 100 | 5.16% | 434 |
| gag | 28 | RELERFAVNP | SF2 | 39 | 66.12% | 435 |
| gag | 93 | KEPFRDYVDR | U455 | 286 | 61.06% | 436 |
| gag | 27 | AEQASQEVKN | IC144 | 303 | 56.69% | 437 |
| gag | 25 | AEQATQEVKN | BZ126B | 304 | 56.69% | 438 |
| pol | 28 | GEAMHGQVDC | U455 | 761 | 66.12% | 439 |
| pol | 41 | REILKEPVHG | IBNG | 462 | 66.12% | 440 |
| pol | 32 | NEQVDKLVSA | SF2 | 700 | 56.69% | 441 |
| pol | 28 | AEHLKTAVQM | U455 | 883 | 56.69% | 442 |
| pol | 33 | EEKIKALVEI | SF2 | 183 | 56.69% | 443 |
| pol | 35 | PEKDSWTVND | U455 | 401 | 48.66% | 444 |
| pol | 29 | IEAEVIPAET | U455 | 798 | 30.65% | 445 |
| pol | 36 | RETKLGKAGY | U455 | 602 | 23.95% | 446 |
| rev | 9 | DEELLKTVRL | MN | 9 | 56.69% | 447 |
| tat | 18 | MEPVDPRLEP | TH475A | 1 | 5.16% | 448 |
| vif | 11 | SESAIRNAIL | JRCSF | 116 | 16.97% | 449 |
| vif | 32 | MENRWQVMIV | U455 | 1 | 5.16% | 450 |
| vpr | 13 | EELKSEAVRH | NL43 | 24 | 65.30% | 451 |
| vpu | 13 | QEELSALVEM | SF2 | 61 | 56.69% | 452 |

TABLE 25
B\hat{ }4006 (9mers) PEPTIDE SEQUENCES

| | | | | | B*4006 | SEQ ID |
|---|---|---|---|---|---|---|
| protein | conservation | sequence | ref. strain | ref. start | (9-mers) | NO: |
| env | 53 | SELYKYKVVE | CAR4054 | 476 | 55.16% | 453 |
| env | 129 | SELYKYKVVK | U455 | 474 | 55.16% | 454 |
| env | 85 | QEVGKAMYAP | SF2 | 425 | 27.31% | 455 |
| env | 64 | LELDKWASLW | SF2 | 660 | 5.69% | 456 |
| env | 117 | FEPIPIHYCA | A_MLY10A | 91 | 1.03% | 457 |
| env | 101 | KEATTTLFCA | SF2 | 45 | 1.03% | 458 |
| env | 100 | TEVHNVWATH | 92UG037.8 | 60 | 1.03% | 459 |
| gag | 48 | AEWDRLHPVH | U455 | 206 | 55.16% | 460 |
| gag | 79 | EEKAFSPEVI | BZ126B | 158 | 27.31% | 461 |
| gag | 76 | TETLLVQNAN | ZAM18 | 261 | 27.31% | 462 |
| gag | 43 | KETINEEAAE | TN243 | 202 | 27.31% | 463 |
| pol | 27 | TELQAIHLAL | SF2 | 632 | 55.16% | 464 |
| pol | 44 | AETFYVDGAA | U455 | 591 | 27.31% | 465 |
| pol | 33 | TEEKIKALVE | SF2 | 182 | 27.31% | 466 |
| pol | 39 | KEKVYLAWVP | SF2 | 683 | 27.31% | 467 |
| pol | 43 | WEFVNTPPLV | U455 | 568 | 12.60% | 468 |
| pol | 36 | TEEAELELAE | U455 | 450 | 9.06% | 469 |
| pol | 38 | TEMEKEGKIS | IBNG | 194 | 5.69% | 470 |
| pol | 44 | LELAENREIL | U455 | 455 | 5.69% | 471 |
| rev | 11 | EELLKTVRLI | MN | 10 | 5.69% | 472 |
| vif | 22 | RDWHLGQGVS | IFA86 | 77 | 2.42% | 473 |
| vif | 32 | MENRWQVMIV | U455 | 1 | 1.03% | 474 |
| vpr | 19 | YETYGDTWAG | SF2 | 47 | 27.31% | 475 |
| vpu | 18 | EELSALVEMG | SF2 | 62 | 5.69% | 476 |

TABLE 26
B\hat{ }4403 PEPTIDE SEQUENCES

| pro- | conser- | | | ref. | | SEQ ID |
|---|---|---|---|---|---|---|
| tein | vation | sequence | ref. strain | start | B*4003 | NO: |
| env | 64 | LELDKWASLW | SF2 | 660 | 22.60% | 477 |
| env | 67 | LEITTHSFNC | SF1703 | 373 | 15.03% | 478 |
| env | 229 | DNWRSELYKY | CA20 | 196 | 11.08% | 479 |
| env | 101 | KEATTTLFCA | SF2 | 45 | 10.03% | 480 |
| env | 68 | GDLEITTHSF | SF1703 | 371 | 8.52% | 481 |
| env | 106 | IEAQQHLLQL | SF2 | 558 | 6.99% | 482 |
| env | 82 | QARVLAVERY | U455 | 570 | 5.31% | 483 |
| gag | 51 | GEIYKRWIIL | BZ126B | 257 | 15.03% | 484 |
| gag | 94 | LGLNKIVRMY | U455 | 264 | 13.83% | 485 |
| gag | 26 | EEQNKSKKKA | SF2 | 106 | 7.87% | 486 |
| gag | 49 | QEVKNWMTET | BNG | 308 | 6.99% | 487 |
| pol | 46 | KEPPFLWMGY | U455 | 377 | 48.34% | 488 |
| pol | 39 | NETPGIRYQY | IBNG | 292 | 48.34% | 489 |
| pol | 29 | AETGQETAYF | U455 | 805 | 43.01% | 490 |
| pol | 43 | RELNKRTQDF | U455 | 232 | 43.01% | 491 |
| pol | 36 | RETKLGKAGY | U455 | 602 | 35.46% | 492 |
| pol | 35 | LEIGQHRTKI | SF2 | 348 | 26.06% | 493 |
| pol | 28 | EPIVGAETFY | SF2 | 587 | 12.02% | 494 |
| pol | 38 | TEMEKEGKIS | IBNG | 194 | 10.03% | 495 |
| rev | 11 | EELLKTVRLI | MN | 10 | 17.14% | 496 |
| tat | 10 | QPKTACTNCY | HXB2R | 17 | 4.01% | 497 |
| vif | 9 | GDARLVITTY | LAI | 60 | 19.96% | 498 |
| vif | 7 | GDAKLVITTY | SF2 | 60 | 19.96% | 499 |
| vpr | 20 | EDQGPQREPY | U455 | 6 | 12.02% | 500 |
| vpu | 15 | IAIVVWTIVF | CDC42 | 18 | 6.61% | 501 |

TABLE 27
B\hat{ }5101 PEPTIDE SEQUENCES

| pro- | conser- | | | ref. | | SEQ ID |
|---|---|---|---|---|---|---|
| tein | vation | sequence | ref. strain | start | B*5101 | NO: |
| env | 85 | LPCRIKQIIN | SF1703 | 421 | 90.57% | 502 |
| env | 100 | CPKVSFEPIP | U455 | 203 | 86.77% | 503 |
| env | 53 | VAEGTDRVIE | SF2B13 | 819 | 78.20% | 504 |
| env | 84 | APTKAKRRVV | Z321 | 497 | 74.67% | 505 |
| env | 58 | APTRAKRRVV | U455 | 490 | 72.16% | 506 |
| env | 72 | GPCKNVSTVQ | SF1703 | 243 | 69.54% | 507 |
| env | 56 | GPCTNVSTVQ | KENYA | 235 | 66.81% | 508 |
| gag | 54 | NPPIPVGEIY | BZ126B | 251 | 83.21% | 509 |
| gag | 26 | NPPIPVGDIY | U455 | 249 | 83.21% | 510 |
| gag | 63 | NANPDCKTIL | VI415 | 325 | 69.27% | 511 |
| gag | 96 | SPRTLNAWVK | UG268 | 143 | 66.81% | 512 |
| pol | 27 | FPISPIETVP | U455 | 154 | 78.42% | 513 |
| pol | 35 | LPEKDSWTVN | U455 | 400 | 76.12% | 514 |
| pol | 29 | WASQIYAGIK | U455 | 420 | 66.53% | 515 |
| pol | 27 | TAVQMAVFIH | U455 | 888 | 63.70% | 516 |
| pol | 43 | QGWKGSPAIF | IBNG | 306 | 63.12% | 517 |
| pol | 28 | SGYIEAEVIP | U455 | 795 | 63.12% | 518 |
| pol | 32 | QPDKSESELV | SF2 | 664 | 49.02% | 519 |
| pol | 43 | GPKVKQWPLT | U455 | 172 | 49.02% | 520 |
| rev | 23 | LPPLERLTLD | SF2 | 75 | 53.90% | 521 |
| tat | 14 | GPKESKKKVE | SF170 | 83 | 74.67% | 522 |
| vif | 14 | DPDLADQLIH | IBNG | 99 | 94.14% | 523 |
| vif | 10 | DPGLADQLIH | SF2 | 99 | 94.14% | 524 |
| vpr | 20 | EAVRHFPRIW | LAI | 29 | 81.01% | 525 |
| vpu | 6 | QPLVILAIVA | TZ023 | 2 | 72.16% | 526 |

TABLE 28
B\hat{ }5102 (9mers) PEPTIDE SEQUENCES

| | | | | | | SEQ |
|---|---|---|---|---|---|---|
| pro- | conser- | | | ref. | B*5102 | ID |
| tein | vation | sequence | ref. strain | start | (9-mers) | NO: |
| env | 84 | APTKAKRRVV | Z321 | 497 | 17.61% | 527 |
| env | 58 | APTRAKRRVV | U455 | 490 | 17.61% | 528 |
| env | 85 | LPCRIKQIIN | SF1703 | 421 | 17.61% | 529 |
| env | 128 | KPVVSTQLLL | U455 | 250 | 11.65% | 530 |
| env | 94 | RPVVSTQLLL | Z321 | 253 | 11.65% | 531 |
| env | 72 | GPCKNVSTVQ | SF1703 | 243 | 7.17% | 532 |
| env | 56 | GPCTNVSTVQ | KENYA | 235 | 7.17% | 533 |
| gag | 54 | NPPIPVGEIY | BZ126B | 251 | 13.33% | 534 |
| gag | 26 | NPPIPVGDIY | U455 | 249 | 13.33% | 535 |
| gag | 63 | NANPDCKTIL | VI415 | 325 | 5.91% | 536 |
| gag | 28 | NANPDCKSIL | U455 | 321 | 4.92% | 537 |
| pol | 27 | FPISPIETVP | U455 | 154 | 56.10% | 538 |
| pol | 27 | TAVQMAVFIH | U455 | 888 | 25.48% | 539 |
| pol | 43 | QGWKGSPAIF | IBNG | 306 | 17.61% | 540 |
| pol | 28 | SGYIEAEVIP | U455 | 795 | 15.37% | 541 |
| pol | 45 | KPGMDGPKVK | IBNG | 168 | 13.33% | 542 |
| pol | 26 | GGIGGFIKVR | U455 | 103 | 8.21% | 543 |
| pol | 29 | WASQIYAGIK | U455 | 420 | 4.92% | 544 |
| pol | 45 | KGIGGNEQVD | U455 | 694 | 3.33% | 545 |
| rev | 23 | LPPLERLTLD | SF2 | 75 | 1.44% | 546 |
| tat | 14 | GPKESKKKVE | SF170 | 83 | 6.01% | 547 |
| vif | 9 | IPLGDARLVI | LAI | 57 | 28.77% | 548 |
| vif | 8 | IPLGDAKLVI | SF2 | 57 | 28.77% | 549 |
| vpr | 20 | EAVRHFPRIW | LAI | 29 | 48.56% | 550 |
| vpu | 6 | QPLVILAIVA | TZ023 | 2 | 22.94% | 551 |

TABLE 29
B\hat{ }5801 (10mers) PEPTIDE SEQUENCES

| | | | | | B*5801 | SEQ |
|---|---|---|---|---|---|---|
| pro- | conser- | | | ref. | (10- | ID |
| tein | vation | sequence | ref. strain | start | mers) | NO: |
| env | 189 | VTVYYGVPVW | U455 | 34 | 72.75% | 552 |
| env | 109 | ITQACPKVSF | U455 | 199 | 68.83% | 553 |
| env | 129 | HSFNCGGEFF | U455 | 372 | 65.14% | 554 |
| env | 86 | HSFNCRGEFF | D687 | 259 | 65.14% | 555 |
| env | 93 | VSFEPIPIHY | U455 | 206 | 53.52% | 556 |
| env | 102 | ITLPCRIKQI | 92UG037.8 | 406 | 48.46% | 557 |
| env | 51 | CSGKLICTTA | SF2 | 597 | 47.67% | 558 |
| gag | 53 | TSTLQEQIGW | K31 | 184 | 71.24% | 559 |
| gag | 42 | ETINEEAAEW | TN243 | 203 | 60.34% | 560 |
| gag | 40 | DTINEEAAEW | U455 | 199 | 60.34% | 561 |
| gag | 36 | PSHKGRPGNF | BZ126B | 437 | 50.55% | 562 |
| pol | 26 | VSAGIRKVLF | SF2 | 707 | 68.83% | 563 |
| pol | 41 | WTYQIYQEPF | U455 | 491 | 68.83% | 564 |
| pol | 45 | STKWRKLVDF | U455 | 222 | 66.78% | 565 |
| pol | 35 | SSMTKILEPF | U455 | 316 | 66.78% | 566 |
| pol | 47 | QATWIPEWEF | U455 | 561 | 62.44% | 567 |
| pol | 45 | NTPPLVKLWY | U455 | 572 | 58.51% | 568 |
| pol | 48 | MGYELHPDKW | U455 | 384 | 54.50% | 569 |
| pol | 40 | ISKIGPENPY | U455 | 201 | 51.73% | 570 |
| rev | 35 | QARRNRRRRW | SF2 | 36 | 65.96% | 571 |
| tat | 9 | FTKKGLGISY | OYI | 38 | 53.52% | 572 |
| vif | 9 | DARLVITTYW | LAI | 61 | 57.54% | 573 |
| vif | 7 | DAKLVITTYW | SF2 | 61 | 57.54% | 574 |
| vpr | 20 | EAVRHFPRIW | LAI | 29 | 53.52% | 575 |
| vpu | 10 | VAAIIAIVVW | SC | 14 | 70.30% | 576 |

TABLE 30
Cw\hat{ }0102 PEPTIDE SEQUENCES

| | | | | | | SEQ |
|---|---|---|---|---|---|---|
| pro- | conser- | | | ref. | Cw* | ID |
| tein | vation | sequence | ref. strain | start | 0102 | NO: |
| env | 54 | NAKTIIVQLN | SF1703 | 286 | 42.05% | 577 |
| env | 66 | TLPCRIKQII | 92UG037.8 | 407 | 42.05% | 578 |
| env | 117 | CAPAGFAILK | U455 | 216 | 19.96% | 579 |
| env | 91 | QLQARVLAVE | U455 | 568 | 19.96% | 580 |
| env | 152 | LTVWGIKQLQ | U455 | 561 | 12.22% | 581 |
| env | 106 | EAQQHLLQLT | US1 | 562 | 12.22% | 582 |
| env | 142 | QLLSGIVQQQ | U455 | 536 | 12.22% | 583 |
| gag | 36 | IWPSHKGRPG | BZ126B | 435 | 42.05% | 584 |
| gag | 66 | RAPRKKGCWK | U455 | 400 | 12.22% | 585 |
| gag | 50 | TLQEQIGWMT | K31 | 186 | 12.22% | 586 |
| gag | 45 | FLQSRPEPTA | SF2 | 450 | 12.22% | 587 |
| pol | 29 | KALTEVIPLT | SF2 | 442 | 42.05% | 588 |
| pol | 28 | NLKTGKYARM | SF2 | 503 | 12.22% | 589 |
| pol | 32 | GAANRETKLG | U455 | 598 | 12.22% | 590 |
| pol | 47 | WVPAHKGIGG | U455 | 689 | 12.22% | 591 |
| pol | 32 | LEPFRKQNPD | SF2 | 323 | 12.22% | 592 |
| pol | 39 | KEPVHGVYYD | IBNG | 466 | 6.87% | 593 |
| pol | 44 | ELAENREILK | U455 | 456 | 6.87% | 594 |
| pol | 43 | GGNEQVDKLV | U455 | 697 | 6.87% | 595 |
| rev | 9 | ILVESPTVLE | LAI | 102 | 6.87% | 596 |
| tat | 6 | DSQTHQASLS | SF2 | 61 | 12.22% | 597 |
| vif | 11 | PLPSVKKLTE | U455 | 162 | 42.05% | 598 |
| vif | 25 | HTGERDWHLG | IBNG | 73 | 6.87% | 599 |
| vpr | 25 | QAPEDQGPQR | U455 | 3 | 6.87% | 600 |
| vpu | 19 | ILRQRKIDRL | CM240X | 33 | 6.87% | 601 |

TABLE 31
Cw\hat{ }0702 PEPTIDE SEQUENCES

| | | | | | | SEQ |
|---|---|---|---|---|---|---|
| | | | | | | ID |
| protein | conservation | sequence | ref. strain | ref. start | Cw*0702 | NO: |
| env | 50 | KYWWNLLQYW | LAI | 799 | 71.91% | 602 |
| env | 83 | LRSLCLFSYH | SF1703 | 765 | 68.10% | 603 |
| env | 81 | ARVLAVERYL | U455 | 571 | 59.94% | 604 |
| env | 58 | SYHRLRDLLL | DA_MAL | 770 | 5.24% | 605 |
| env | 146 | FNCGGEFFYC | P104 | 105 | 4.95% | 606 |
| env | 93 | IRPVVSTQLL | Z321 | 252 | 3.38% | 607 |
| env | 58 | IRQGLERALL | U455 | 847 | 3.18% | 608 |
| gag | 32 | LRPGGKKKYR | BNG | 21 | 99.90% | 609 |
| gag | 31 | LYNTVATLYC | K7 | 78 | 94.28% | 610 |
| gag | 74 | FSPEVIPMFS | U455 | 160 | 16.37% | 611 |
| gag | 71 | IRQGPKEPFR | U455 | 281 | 9.78% | 612 |
| pol | 44 | TPPLVKLWYQ | U455 | 573 | 74.16% | 613 |
| pol | 26 | KRKGGIGGYS | U455 | 900 | 70.51% | 614 |
| pol | 46 | IYQYMDDLYV | U455 | 334 | 46.95% | 615 |
| pol | 46 | EPPFLWMGYE | U455 | 378 | 37.86% | 616 |
| pol | 46 | TVLDVGDAYF | U455 | 261 | 27.09% | 617 |
| pol | 42 | QYALGIIQAQ | U455 | 654 | 25.31% | 618 |
| pol | 40 | LKEPVHGVYY | IBNG | 465 | 19.97% | 619 |
| pol | 34 | KQGQGQWTYQ | SF2 | 486 | 17.05% | 620 |
| rev | 22 | LQLPPLERLT | SF2 | 73 | 2.99% | 621 |
| tat | 7 | LNKGLGISYG | UG275A | 39 | 24.44% | 622 |
| vif | 6 | QYLALAALIK | NL43 | 146 | 17.40% | 623 |
| vif | 6 | QYLALAALIT | SF2 | 146 | 17.40% | 624 |
| vpr | 10 | LHGLGQHIYE | IBNG | 39 | 21.14% | 625 |
| vpu | 11 | VWTIVFIEYR | CDC42 | 22 | 1.78% | 626 |

The details of one or more embodiments of the invention are set forth in the accompanying description above. Although any methods and materials similar or equivalent to those described herein can be used in the practice or testing of the present invention, the preferred methods and materials have been described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but only to the claims appended hereto.

We claim:

1. A vaccine, comprising:
a human immunodeficiency virus (HIV) vaccine candidate peptide containing an amino acid sequence selected from the group of the sequences consisting of SEQ ID NOS:1-31, 33-85, 87-109, and 111-672, in an immunologically acceptable excipient.

2. The vaccine of claim 1, wherein the peptide is between 8 amino acids and 50 amino acids in length.

3. The vaccine of claim 1, wherein the HIV vaccine candidate peptide has an amino acid sequence selected from the group of the sequences SEQ ID NO:1-31, 33-85, 87-109, and 111-672.

4. The vaccine of claim 1, wherein the peptide is complexed to a carrier protein.

5. The vaccine of claim 1, wherein the peptide is a recombinant fusion protein.

6. The vaccine of claim 1, wherein the excipient is an adjuvant.

7. A recombinant human immunodeficiency virus (HIV) vaccine candidate peptide, comprising:
a peptide containing an amino acid sequence selected from the group of the sequences SEQ ID NO:1-31, 33-85, 87-109, and 111 -672, wherein the peptide is expressed from a recombinant polynucleotide.

8. The recombinant peptide of claim 7, wherein the recombinant polynucleotide is a naked DNA vaccine.

9. A method for inducing an anti-human immunodeficiency virus (anti-HIV) immune response, comprising:
administering to a mammalian subject a HIV vaccine candidate peptide containing an amino acid sequence selected from the group of the sequences SEQ ID NOS:1-31, 33-85, 87-109, and 111-672.

10. The method of claim 9, wherein the induction of an anti-HIV immune response is the raising of an anti-HIV antibody.

11. The method of claim 9, wherein the mammalian subject is a human.

12. The method of claim 9, wherein the administration is selected from the group consisting of orally, topically, parenterally, by viral infection, and intravascularly.
